(19) United States
(12) Patent Application Publication
(10) Pub. No.: US 2008/0140110 A1
(43) Pub. Date: Jun. 12, 2008

(54) IMPLANT, SYSTEMS AND METHODS FOR PHYSICALLY DIVERTING MATERIAL IN BLOOD FLOW AWAY FROM THE HEAD

(76) Inventor: Paul A. Spence, Louisville, KY (US)

Correspondence Address: WOOD, HERRON & EVANS, LLP, 2700 CAREW TOWER, 441 VINE STREET, CINCINNATI, OH, 45202, US

(21) Appl. No.: 11/954,438

(22) Filed: Dec. 12, 2007

Related U.S. Application Data
(60) Provisional application No. 60/869,610, filed on Dec. 12, 2006.

(51) Int. Cl. A61M 29/00; A61F 2/06

(52) U.S. Cl. 606/200; 623/1.1

(57) ABSTRACT

A device for preventing stroke due to embolic material in the bloodstream of a patient, the patient having an aorta with an ascending portion and a descending portion, and one or more arch vessels communicating with the aorta for directing blood flow to the brain of the patient. The device includes a physical deflector element configured for at least partial placement in the aorta of the patient and a mounting structure coupled to the physical deflector element. The mounting structure is configured to engage at least one of the aorta or an arch vessel communicating with the aorta. The physical deflector element is constructed and arranged to direct blood flow in the aorta in a manner that directs embolic material in the blood flow past the one or more arch vessels and into the descending portion of the aorta.

This application claims the benefit of U.S. Provisional Patent Application Ser. No. 60/869,610, filed Dec. 12, 2006 (pending), the disclosure of which is also fully incorporated by reference.

### TECHNICAL FIELD

The present invention generally relates to stroke prevention and, more particularly, to apparatus and methods for preventing material, such as particulates or air bubbles, from traveling into arteries leading to the head of a patient.

### BACKGROUND

Stroke is a major cause of death and disability worldwide. In 2002, there were 700,000 patients in the United States who suffered a new or recurrent stroke and 162,000 of these patients died. It is estimated that the cost of stroke in the U.S. alone is 57 billion dollars per year. Patients and their families fear strokes because of the significant levels of permanent disability strokes can produce. Many patients are rendered immobile, non-functional and/or unable to communicate due to strokes.

Strokes occur due to disruption of blood flow to the brain. This can occur due to occlusion of vessels or with obstruction of vessels by an embolus that lodges in an important vessel perfusing the brain with blood. An embolus is a material that travels in the blood circulation to a distant location and one of the most common origins for an embolus is the heart. Atrial fibrillation is an irregular heart rhythm during which the atrial chambers do not empty themselves of blood to the same extent as a heart in a normal rhythm. In this situation, the more stagnant pool of blood remaining in the atrial chambers can form clots and these clots can dislodge and embolize with the potential for then traveling into the brain. Approximately one third of all strokes are due to emboli that occur in patients who have atrial fibrillation.

There are many sources of emboli that may travel into the brain. For example, clots or other material can travel from any part of the heart. The left ventricle can develop clots particularly after myocardial infarction or when the heart is enlarged and segments of the heart are not moving properly. Heart valves may also give rise to clot or infective material that may travel to the brain. Artificial or replacement heart valves can also develop clots that embolize. Defects in the heart walls, such as in an atrium or ventricle, can allow clots to travel from leg veins through the heart and into the brain (i.e., paradoxic emboli). Emboli can also arise from the aorta, such as emboli resulting from atherosclerotic disease of the ascending aorta.

Once an emboli is in position within the brain for more than three to four hours, much of the damage to the brain becomes permanent. Because the brain is very unforgiving of decreased blood flow, it would very useful for doctors to have therapy to prevent the occurrence of a stroke. Such a therapy could be applied in high risk patients, including those, for example, who experience atrial fibrillation or who have already suffered from one or more previous stroke incidents.

Perfusion of blood into the brain arises from the three arch vessels in the aorta. These arch vessels arise on the outer curvature of the aortic arch above the heart. This is the curved portion of the aorta connecting the ascending aorta to the descending aorta. Unfortunately, since these arch vessels are the first large branches on the aorta and are located on the outside of the turn or curve in the aorta, material within the blood flow tends to naturally stream into these arch vessels and lodge in branches inside the brain. Past research studies on animals demonstrated that metal pellets introduced in the heart consistently lodge in vessels perfusing the brain.

Since the risk of stroke in a typical untreated atrial fibrillation patient is only 8% each year, a treatment must be easy to perform and reliable and must not interfere with the lifestyle of the patient. Thus, there are advantages to treatments that do not require any external power source or recharging device. Additionally, it would be desirable to provide treatments that can solve the problems of emboli arising from anywhere in the heart, ascending aorta, arch of the aorta or elsewhere in the body.

### SUMMARY

In various embodiments, the present invention is generally directed to a device for preventing stroke due to embolic material in the blood stream of a patient. The device can generally comprise a physical deflector element configured for at least partially placement in the aorta of the patient. Mounting structure is coupled to the physical deflector element and is configured to engage at least one of the aorta or an arch vessel communicating with the aorta. The physical deflector element is constructed and arranged to direct blood flow in the aorta in a manner that directs embolic material in the blood flow past the one or more arch vessels and into the descending portion of the aorta.

A method of physically directing embolic material in blood flow within the aorta may include mounting a physical deflector element at least partially within the aorta. The physical deflector element is then used to direct a first portion of the blood flow past an entrance of the arch vessel. A second portion of the blood flow is directed to the entrance of the arch vessel.

Another method involves replacing a portion of the aorta with a tubular aortic graft having at least one tubular arch vessel graft coupled thereto. The method can comprise replacing a portion of the aorta with the tubular aortic graft such that the tubular arch vessel graft is misaligned with an arch vessel of the patient. The misaligned tubular arch vessel graft is then connected with the arch vessel.

Various embodiments involve the placement of stroke prevention tubular devices partially in the arch vessels such that one or more portions thereof extend into the aorta. These may be constructed as stent-like expandable devices in many different manners.

The invention also generally provides a system for preventing stroke. The system includes at least catheter device used to deliver the physical deflector element and/or the mounting structure to the aorta and/or to one of the arch vessels.

Additional features and aspects will become more readily apparent to those of ordinary skill upon review of the illustrative embodiments and the drawings associated therewith.

### BRIEF DESCRIPTION OF THE DRAWINGS

FIG. 1 is a schematic view of a patient undergoing a catheter-based procedure in accordance with one embodiment of the invention.

FIG. 2A is an enlarged view of the aorta and of the stroke prevention device of FIG. 1.

FIG. 2B is a cross sectional view of the aorta and of a stroke prevention device according to another embodiment.

FIG. 2C is a view similar to FIG. 2B, but illustrating another alternative embodiment of a stroke prevention device.

FIG. 2D is a cross sectional view similar to FIG. 2C, but illustrating another alternative embodiment.

FIG. 2E is a cross sectional view similar to FIG. 2D, but illustrating another alternative embodiment.

FIG. 2F is a cross sectional view similar to FIG. 2E, but illustrating another alternative embodiment.

FIG. 3 is another cross sectional view similar to FIGS. 2A-2E, but illustrating another alternative embodiment.

FIG. 3A is a cross sectional view taken along line 3A-3A of FIG. 3.

FIG. 4 is a cross sectional view of the aorta, and illustrating another alternative embodiment of a stroke prevention device.

FIG. 4A is a perspective view illustrating a deflector element of FIG. 4.

FIG. 5 is a cross sectional view of the aorta illustrating perspective views of deflector elements secured partially within the arch vessels.

FIGS. 5A-5E illustrate various alternative embodiments of deflector elements securable within an arch vessel.

FIG. 6 is a cross sectional view similar to FIG. 5, but illustrating an alternative embodiment of a stroke prevention device.

FIG. 7A is a cross sectional view similar to FIG. 6, but illustrating another alternative embodiment.

FIG. 7B is a cross sectional view similar to FIG. 6, but illustrating another alternative embodiment.

FIG. 7C is a cross sectional view similar to FIG. 6, but illustrating another alternative embodiment.

FIG. 8A is a cross sectional view of the aorta illustrating another alternative stroke prevention device.

FIG. 8B is a perspective view of the device illustrated in FIG. 8A.

FIG. 8C is a perspective view similar to FIG. 8B, but illustrating an alternative configuration.

FIG. 9A is a cross sectional view of the aorta and illustrating another alternative embodiment of a stroke prevention device.

FIG. 9B is a cross sectional view similar to FIG. 9A, but illustrating an alternative embodiment of the device.

FIG. 10A is a cross sectional view of the aorta illustrating another alternative stroke prevention device.

FIG. 10B is a cross sectional view taken along line 10B-10B of FIG. 10A.

FIG. 11A is a cross sectional view of the aorta illustrating an alternative embodiment of a stroke prevention device.

FIG. 11B is a view similar to FIG. 11A, but illustrating an alternative embodiment.

FIG. 12 is a cross sectional view of the aorta illustrating a stroke prevention device according to another alternative embodiment.

FIG. 13A is a cross sectional view of the aorta illustrating a stroke prevention device according to another alternative embodiment.

FIG. 13B is a cross sectional view taken along line 13B-13B of FIG. 13A.

FIG. 14A is a cross sectional view of the aorta illustrating a stroke prevention device according to another alternative embodiment.

FIG. 14B is a cross sectional view taken along line 14B-14B of FIG. 14A.

FIG. 15 is a cross sectional view of the aorta illustrating a blood flow profile in schematic fashion.

FIG. 16 is a view similar to FIG. 15, but illustrating flow characteristics with lighter and darker blood flow regions.

FIG. 17 is a cross sectional view of an aortic graft according to one embodiment.

FIG. 18 is a cross sectional view of an aortic graft according to another embodiment.

FIG. 19 is a cross sectional view of an aortic graft according to another alternative embodiment.

FIG. 20 is a cross sectional view of an aortic graft according to another alternative embodiment.

FIG. 21 is a cross sectional view of an aortic graft according to another alternative embodiment.

FIG. 22 is a cross sectional view of an aortic graft according to another alternative embodiment.

FIG. 23 is a cross sectional view of the aorta with another alternative stroke prevention device.

FIG. 24 is a cross sectional view of the aorta with another alternative stroke prevention device.

FIGS. 25A and 25B schematically illustrate the insertion of a stroke prevention device.

FIG. 26 is a cross sectional view of the aorta and demonstrating the use of an embolic protection device.

FIG. 27 is a cross sectional view of the aorta illustrating another embolic protection device.

FIG. 28A is a perspective view of an aortic graft with embolic protection devices coupled therewith.

FIG. 28B is a perspective view illustrating containment of the device shown in FIG. 28A in a catheter.

### DETAILED DESCRIPTION OF THE ILLUSTRATIVE EMBODIMENTS

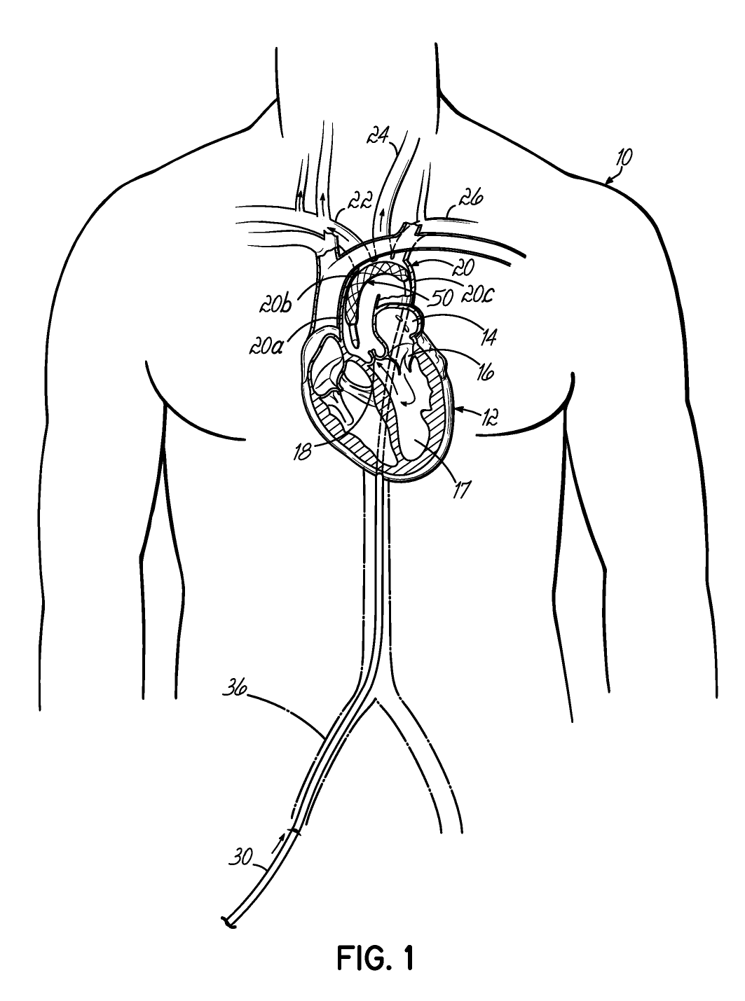

Like reference numerals in the drawings refer to identical elements and, therefore, for purposes of brevity these elements may not be specifically mentioned or described in the later portions of the written description. FIG. 1 is an illustration of a patient 10 with the heart 12 of the patient 10 in cross section. A catheter 30 is shown to be directed through an artery 36 in the groin region, such as the femoral or iliac area, and carries a physical deflector device 50 shown to be in an unexpanded or contracted state at a distal end of the catheter 30. The heart 12 receives blood flow from the left atrium 14 through the mitral valve 16 into the left ventricle 17. The blood then is pumped through the aortic valve 18 into the aorta 20. The aorta 20 includes an ascending portion 20a, an arch or curved portion 20b, and a descending portion 20c. Three arch vessels 22, 24, 26 take off from the aorta 20 generally at the arch 20b. Blood flow through these three arch vessels 22, 24, 26 directs oxygenated blood to the brain and upper extremities of the patient. Various examples of emboli deflectors will be shown and described herein and each may be used alternatively in a permanent fashion or a temporary fashion in any particular patient. Temporary uses, for example, may be desirable in situations where emboli deflection may be necessary only during a medical procedure, such as any procedure having a risk of dislodging material into the bloodstream. It will also be appreciated that the deflector device or devices 50, and any of the other devices described herein may be introduced in a minimally invasive manner into the arterial tree via a branch of an artery or a puncture into an artery anywhere in the patient's body. The deflector devices described herein may be implanted instead in an open surgical operation, or at any level of less invasive procedures, including robotic approaches, minimally invasive procedures and keyhole procedures. With regard to the use of catheters, it will be appreciated that any catheter introduction procedures may be followed, including the use or uses of guide wires to facilitate positioning the catheter, such as typical over-the-wire techniques, and catheter delivery devices that allow the deflector device 50 to be initially collapsed during introduction into the endovascular system and then activated and expanded when positioned properly. The deflector may have any suitable components for holding or mounting the deflector device 50 in place within the aorta, such as stents, hooks or spring-like biasing elements.

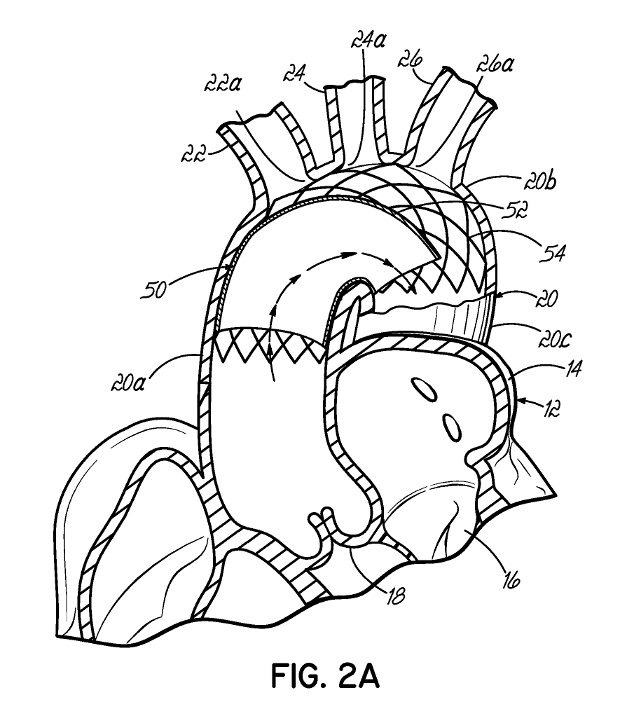

FIG. 2A illustrates an enlarged cross sectional view of the upper portions of the heart 12, the curved area or arch 20b of the aorta, and the three arch vessels 22, 24, 26. The figure further illustrates an expanded deflector device 50 that physically deflects or channels embolic material traveling from or through the heart 12, through the aortic valve 18 and the aorta 20 into the downwardly directed or descending portion 20c of the aorta 20 downstream from the entrance 22a, 24a, 26a to each of the arch vessels 22, 24, 26. It should be noted that the anatomy illustrated in the drawings is simplified for clear illustration purposes. Various portions of the illustrated anatomy, such as arch vessel entrances 22a, 24a, 26a, actually have more complicated features and detail. Material in the bloodstream, such as solid or gaseous material, will have a strong tendency to enter the brain via the arch vessels 22, 24, 26. The innominate artery 22 (giving rise to the right carotid and right subclavian), the left carotid artery 24, and the left subclavian artery 26 all have a relatively direct path of flow from the heart 12 and, therefore, material in the bloodstream from the heart 12 will tend to enter the brain through these vessels. The deflector device 50 forces the blood flow to pass by the arch vessels 22, 24, 26, however, blood will still flow to the brain because the blood will pass through a tube 52 within the deflector device 50 and turn back towards the arch vessels 22, 24, 26 in a retrograde manner. On the other hand, embolic materials within the blood flow will be less likely to change direction and more likely to continue on a path downward to the lower portions of the body. Organs other than the brain are much more forgiving when they encounter an embolus. For example, an embolus is much less dangerous when entering the legs or the kidneys.

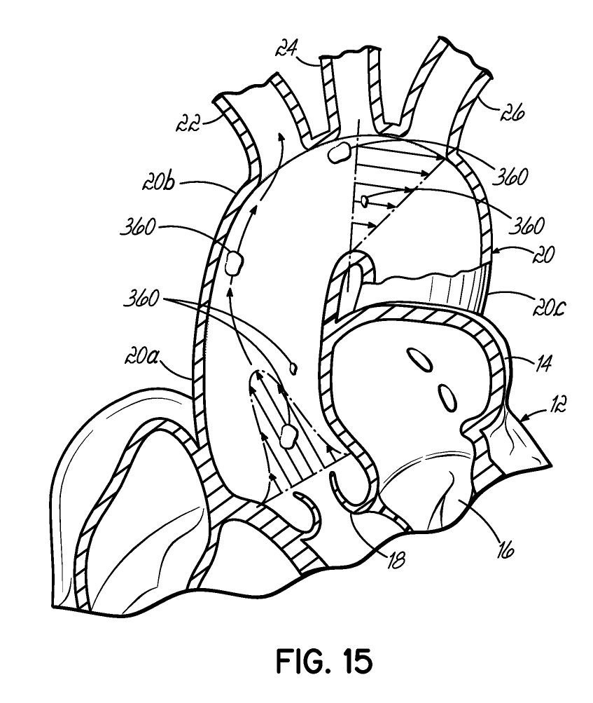

The deflector 50 shown in FIG. 2A may be formed in various lengths and it may cover or extend within various lengths of the aorta 20. Blood flow to the brain through the arch vessels 22, 24, 26 should not be restricted by the deflector device 50 and the deflector tube 52 should not be too small such that it obstructs the blood flow to the distal aorta by creating a high pressure gradient. The deflector device 50 may be formed in a manner similar to a stent mounted tube. A wire mesh type mounting 54 is shown, but the mounting may also be a coil as opposed to a wire mesh type stent, or may have any other suitable alternative or additional mounting features. Various types of aortic stent grafts may be used, including those that have a zig zag form of wire or other semi-rigid support structure. For example, such support structures are shown in grafts in FIGS. 18, 19 and 20 of the present application. Suitable aortic stent grafts are obtainable from companies such as W.L. Gore and Associates, Inc., Medtronic, Inc. and Cook Group, Inc. The deflector device 50 may be formed from any biocompatible material, such as plastics or metals such as stainless steel or Nitinol. The tube may also be any biocompatible material including fabrics, such as Dacron, Teflon, Gortex, etc. or biologic materials such as bovine, pericardium or tissue engineered materials. The chosen materials may include clot resistance features and design characteristics to prevent areas of high shear stress or stasis of blood flow. Various coatings and surface treatments (such as roughening) may be used, as appropriate, to encourage tissue ingrowth on those areas of the implant that can benefit from such a feature. An overgrown surface is much less likely to clot as it presents a biologic surface to the blood. Coatings that prevent formation of clots, protein build up, etc. may be used as well. This could include a variety of anticoagulants such as Heparin and other clot repelling agents.

FIG. 2B illustrates another possible configuration for a deflector device 70 that directs clots or other emboli away from the arch vessels 22, 24, 26. The device 70 is again shown as a stent-type device including a physical deflector portion or ramp 72 mounted on a wire mesh or coil 74 of the device 70. Again, the stent-type device 70 may be substituted with or may further include other mounting features for retention purposes, or may be sutured into position during an operation. This deflecting structure 72, like any of the other types of embolus deflecting or redirecting structures referred to herein, may be incorporated into a surgical replacement graft, non-limiting examples of which are illustrated in FIGS. 18-20. Also, barbs or hooks may be used to anchor the device 70 into the aorta 20. One advantage of the device 70 shown in FIG. 2B is that there is no direct cover or physical barrier over the entrances 22a, 24a, 26a of the arch vessels 22, 24, 26 so the risk of obstruction to blood flow will be lower. The height, angle, length, location and pattern (such as a straight slope, curved slope, etc.) of the physical deflector portion 72 may be varied in any suitable manner. The goal with this device 70 is to deflect the emboli 76 such that the material in the bloodstream passes beyond the entrances 22a, 24a, 26a to the arch vessels 22, 24, 26. Practically speaking, most of the blood flow to the brain is derived from the first two arch vessels (i.e., the innominate artery 22 and the left carotid artery 24) and protecting these two vessels will most often be a priority. The deflector device 70 may also have a horseshoe shape that tracks lateral to the arch vessels (not shown). It could also have an oval shape, such that the arch vessels communicate through the center of the oval, annular shape.

FIG. 2C illustrates another alternative deflector device 90 including a physical deflector tube 92 and a stent-type mounting portion 94. The tube 92 includes a generally spiral shaped element 96 for redirecting or urging the blood flow into a generally whirling motion. When blood exits the aortic valve 18, its flow rate is higher at the center of the valve 18 than at radially outward portions of the valve 18 and the aorta 20. The spiral shaped element 96 is shown to have a right hand or clockwise spiral when viewed from the inlet 92a of the tube 92, however, this spiral may be reversed. There is a natural spiral to the flow of blood as it exits the aortic valve 18. When viewed from below, the blood demonstrates a right hand turn. Augmenting this natural spiral flow may be the easiest way to perform this type of embolus deflection or redirection. Material in the blood tends to travel to the center of a spiral or vortex flow and, therefore, imparting a spiral flow to the blood will cause the material or emboli in the blood to be directed toward the center of the flow. Thus, the spiral blood flow encourages material or debris to remain in the center of the aorta 20 rather than passing into the arch vessels 22, 24, 26. The spiral shaped element 96 in conjunction with the curved tubular member 92 help ensure that any embolic particles or material will exit the tube 92 and remain in the center of the aorta 20, and prevent them from turning back toward the entrances 22a, 24a, 26a to the arch vessels 22, 24, 26.

FIG. 2D illustrates another embodiment of a device 100 including a spiral element 102, but without the use of a bypass tube. Instead, the generally spiral shaped element 102 or elements will encourage the blood to continually spiral thus forcing any material or particles to the center of the blood flow within the aorta 20 and away from the arch vessels 22, 24, 26 which connect at the upper side or arch 20b of the aorta 20. The stent-like mounting member 104 could be an open mesh or could have dedicated openings to allow blood flow therethrough into the arch vessels 22, 24, 26.

FIG. 2E illustrates another embodiment of a deflector device 110 including a series of deflectors 102, as opposed to a continuous deflector member, mounted on a stent-like structure 104. The series of deflectors 102 are designed to encourage particles or other embolic material to remain in the center of blood flow through the turn 20b in the aorta 20 and, similar to the previous embodiments, act as “speed bumps” to keep material out of the brain.

FIG. 2F illustrates another embodiment of a deflector device 120 that will encourage a generally spiral flow of blood through the turn 20b of the aorta 20. Here a multi-lumen tube 122 is formed generally in a spiral fashion, and again the spiral may turn or rotate either clockwise or counterclockwise and may be of any desired uniform or non-uniform pitch. As with all other embodiments, this physical deflector portion (e.g., tube 122 in this embodiment) may be mounted in the aorta 20 in any desired manner, although a stent-like expandable mesh element 124 is again shown for illustration. The spiral maintains the particles or material moving downwardly through the turn 20b in the aorta 20 rather than reversing back upwardly and traveling through the arch vessels 22, 24, 26 into the brain. This feature also shows the principle of treating the high risk portion of the blood flow (i.e., the center of the blood flow which perhaps more likely contains the particles or other emboli) and diverting it to the descending and distal aorta 20c downstream of the entrances 22a, 24a, 26a to the arch vessels 22, 24, 26.

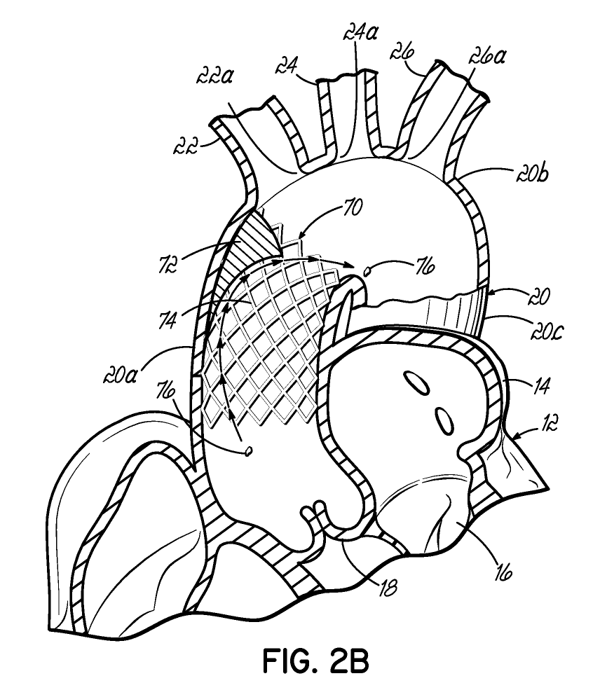

FIG. 3 illustrates another embodiment of a physical deflector device 130 that does not involve the use of a tube within a stent or connected to other mounting structure. Instead, a shield member 132 is coupled to a mounting member 134 which, again, in this illustrative example is a stent-like member, but may be any suitable mounting structure. The shield member 132, as best shown in the cross section of FIG. 3, may be a partial tube structure with an open top portion 132a that may communicate with each of the entrances 22a, 24a, 26a to the arch vessels 22, 24, 26 and which has an open inlet 132b and for receiving a reverse flow of blood as shown by the arrows 136. Particles or other emboli will be less likely to make the reverse turn into the inlet 132b and upwardly into the arch vessels 22, 24, 26. Moreover, if a particle or other emboli 138 do make this turn, they will more likely enter the left subclavian artery 26 first, and less likely to cause brain injury as a result. The shield member 132 should be constructed so as to maintain blood flow through the aorta 20 around the turn 20b to the descending and distal aorta 20c, but still allow adequate blood flow to the brain via the arch vessels 22, 24, 26. The forward or upstream end 132c of the shield member 132 can be closely sealed or fitted to the wall of the aorta 20 upstream of the takeoff or entrance 22a of the first arch vessel 22 so that blood flowing at this location does not pass directly under the shield member 132 and reach the brain while potentially carrying emboli. For example, a flange shaped edge and/or gasketing, biocompatible materials may be used to provide a seal at least at this location 132c of the shield member 132. FIG. 3A, which is a cross section of FIG. 3, shows a tubular structure, however, any other shape may be used, such as flatter shapes or shapes having straight walls as opposed to a continuously curved wall as shown in FIG. 3A. In addition, the tubular structure 132 may contact or cover a larger surface area of the inner wall of the aorta 20 as opposed to covering only the margin, as shown, immediately adjacent to the entrances 22a, 24a, 26a of the arch vessels 22, 24, 26. It may be desirable to promote a seal at least at location 132c, if not along the entire margin of the structure 132 in contact with the aortic tissue. This may be accomplished by maintaining close apposition between structure 132 and aorta 20 and allowing tissue ingrowth which can be facilitated by using porous graft materials, or stent designs, or folded metal designs (e.g., similar to steel wool balls used is atrial septal defect occluders) that promote tissue entry.

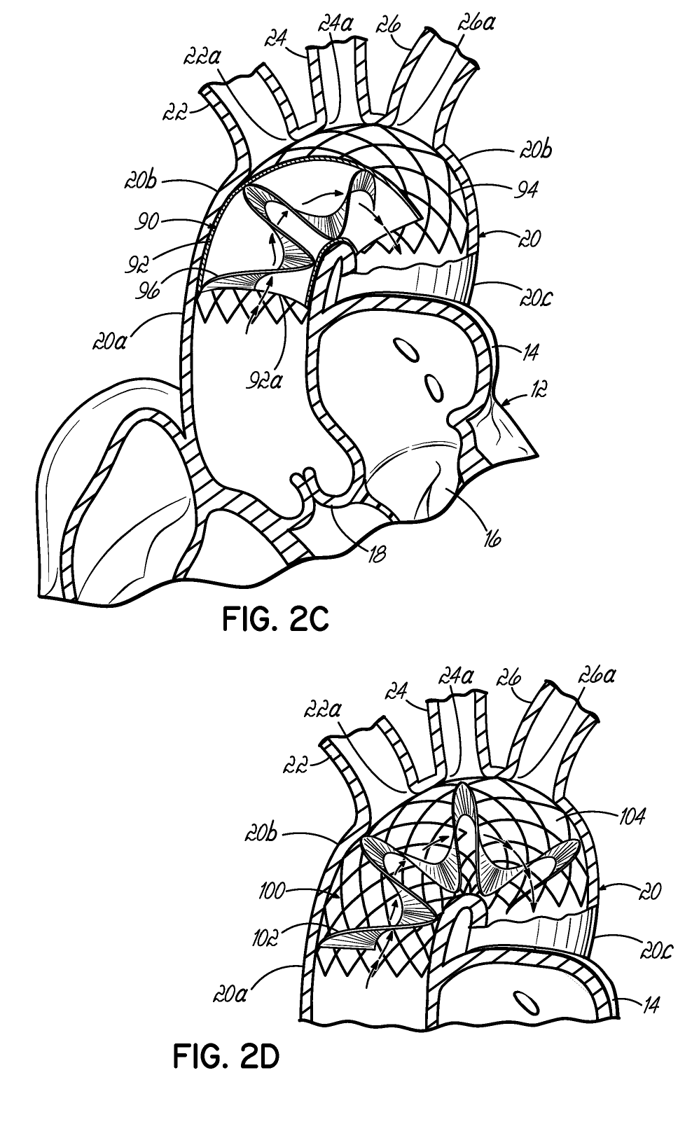

FIGS. 4 and 4A illustrate an embodiment of a physical deflector device 150 that can ensure greater blood flow into the arch vessels 22, 24, 26. In particular, FIG. 4 illustrates a series of deflector elements 152 placed over and adjacent the entrances 22a, 24a, 26a to the arch vessels 22, 24, 26. These deflector elements 152 may have a lower profile than the single “speed bump” deflector and may afford better protection by a sequential “hand-over-hand” series of deflections to keep material out of the brain. The deflector elements 152 are mounted on a stent-like device 154, however, they may be mounted in other ways as would be other embodiments described herein. Many different configurations may be used for the deflector device 150, with FIGS. 4 and 4A showing one potential configuration or shape. If these deflector elements 152 shift in location after implantation, this should not significantly obstruct or interfere with blood flow and the deflector elements 152 may not require alignment with the arch vessels 22, 24, 26. For example, the deflector elements 152 could straddle one or more entrances 22a, 24a, 26a to the arch vessels 22, 24, 26 without obstructing the entrance.

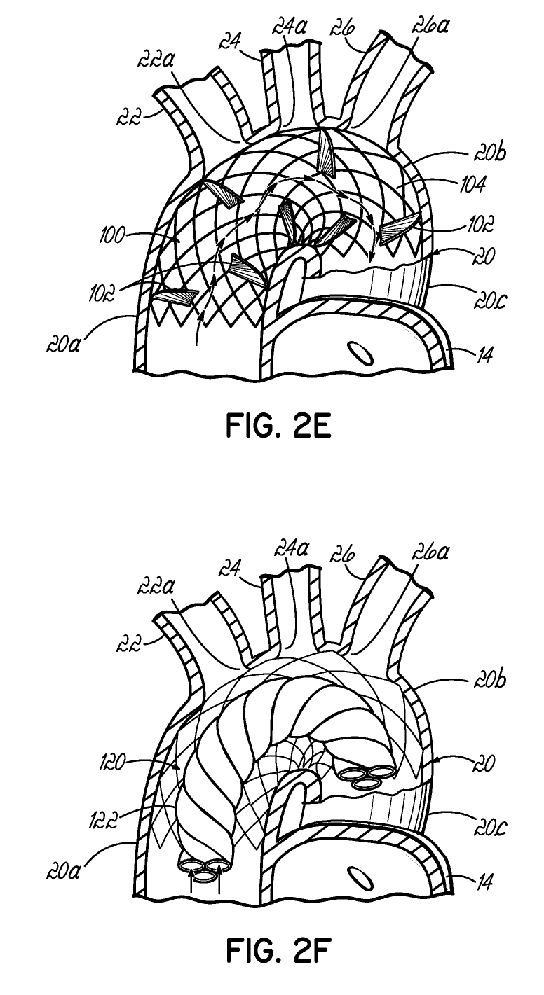

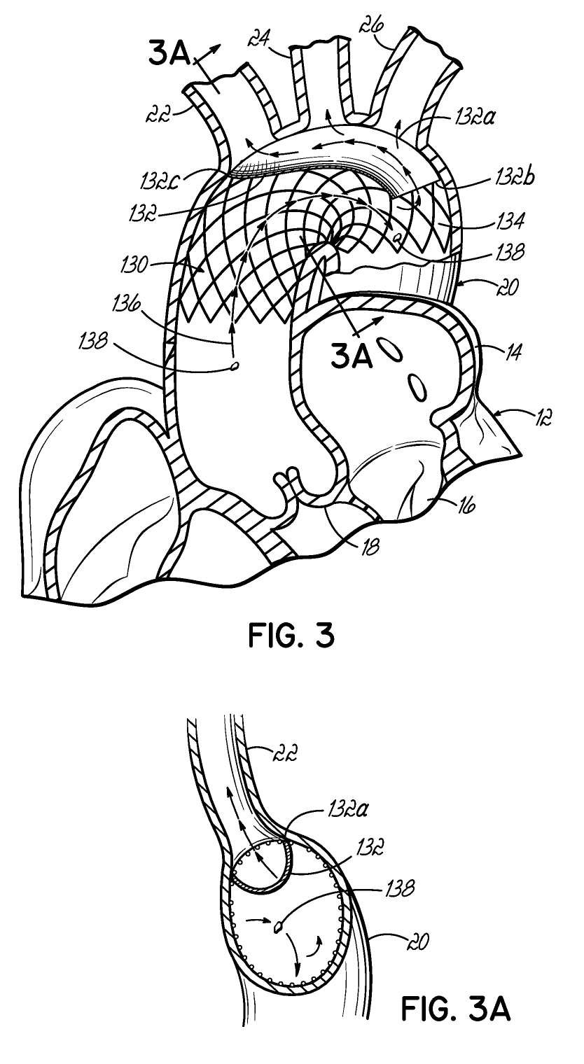

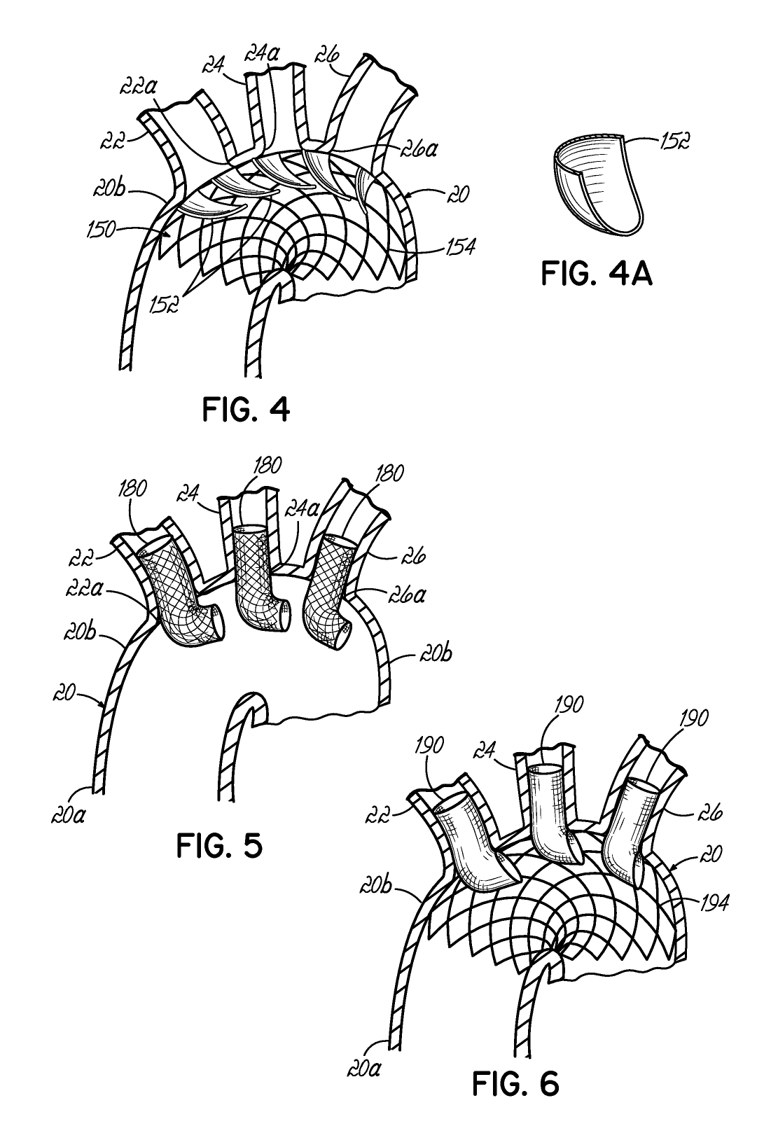

FIGS. 5, 5A-E, 6, 7A and 7B each illustrate various embodiments of individual tubular deflector elements that may be inserted and mounted within each of the respective entrances 22a, 24a, 26a to the arch vessels 22, 24, 26. Each of the tubular elements includes a blood flow entrance and a blood flow exit end. The exit end of each tube extends within the respective arch vessel 22, 24, 26, while the entrance end extends within the aorta 20. A bend in the tube locates the entrance end a suitable or desired distance downstream in the aorta such that particles will tend to be deflected by the tubular member and continue within the blood flow as opposed to reversing direction and entering the entrance end of one of the tubes. One or more of the arch vessels 22, 24, 26 may be protected by the separate tubes, although in the embodiments shown, each of the arch vessels 22, 24, 26 is protected by a separate tube. Separate tubes 180 may be mounted individually as shown in FIG. 5, or tubes 190 may be mounted to a common mounting structure 194, such as a stent-like device as shown in FIG. 6 or in FIG. 7A illustrating tubes 200 and mounting structure 204. The tubes may extend into the aorta from each of the arch vessels with a desired length as shown in these figures. In addition, the entrance ends of the tube may have various shapes, such as the shapes shown by way of example in these figures, or other shapes. The entrance ends of the tube may, for example, be flat, trumpet-shaped, angled or include any variety of other features and shapes. Grooves could be added to the surfaces to preferentially direct blood flow. FIG. 5A shows a tube 210 with a trumpet shaped entrance 210a. FIG. 5B illustrates a tube 212 with an upward curve or angling of the entrance end 212a, however, the curved end could be downward or both upward and downward. FIG. 5C illustrates another embodiment of a tube 214 insertable within an arch vessel for connection therewith as previously described with respect to FIG. 5, for example, but having an inlet or blood entrance 214a that is tapered or reduced in diameter relative to immediately adjacent sections of the tube 214. FIG. 5D is a top view of another tube 216 having a blood entrance end 216a with blood deflector elements or baffle structures 216b for deflecting blood away from the entrance end 216a as the blood flows past the tube 216 when the tube 216 is coupled within an arch vessel as previously described, for example, in connection with FIG. 5. FIG. 5E illustrates another embodiment of a tube 218, similar to that shown in FIG. 5, but including a blood inlet end 218a having a fluted design with concave recesses 218b shown as examples. These flutes or recesses 218b may be used to deflect blood or give beneficial blood flow characteristics as the blood passes the entrance end 218a to further assure that embolic material does not flow into the entrance end 218a and thereby enter one of the arch vessels. One or more of the tubes may extend deeper into the descending aorta as shown with the tubes 230 in FIG. 7B. This configuration would require that blood flows retrograde from the descending aorta 20c to the head and a particle or other emboli would have to make a 180° turn to enter the arch vessels 22, 24, 26. FIG. 7C illustrates that multiple tube portions 240a, 240b, 240c within each of the individual arch vessels 22, 24, 26 may connect together into a single tube 242 having an entrance end 242a that requires retrograde flow. Although not shown, the leading or upstream edge of each tube facing the blood flow may be formed in a more aerodynamic or fluid dynamic manner with an edge or surface that is angled or constructed with a curved shape similar to the bow of a boat. This leading edge may also have grooves or other features that encourage blood to flow past without injury, such as hemolysis caused by impact with a flat or rough surface. Such a design may also encourage the particles or other emboli to pass by the tubes and flow on into the descending aorta 20c. A series of tubes within each arch vessel 22, 24, 26 may be of different individual shapes and sizes as desired or needed for the particular situation. The chosen material for the tubes may again be any biocompatible material such as a metal, nonmetal, combinations of metals and nonmetals, biologic or engineered materials. As with all of the embodiments contemplated herein, it may be desirable to use a material that encourages fibrous ingrowth such that the foreign object (i.e., the tubes or other deflector members) become part of the patient's natural tissue.

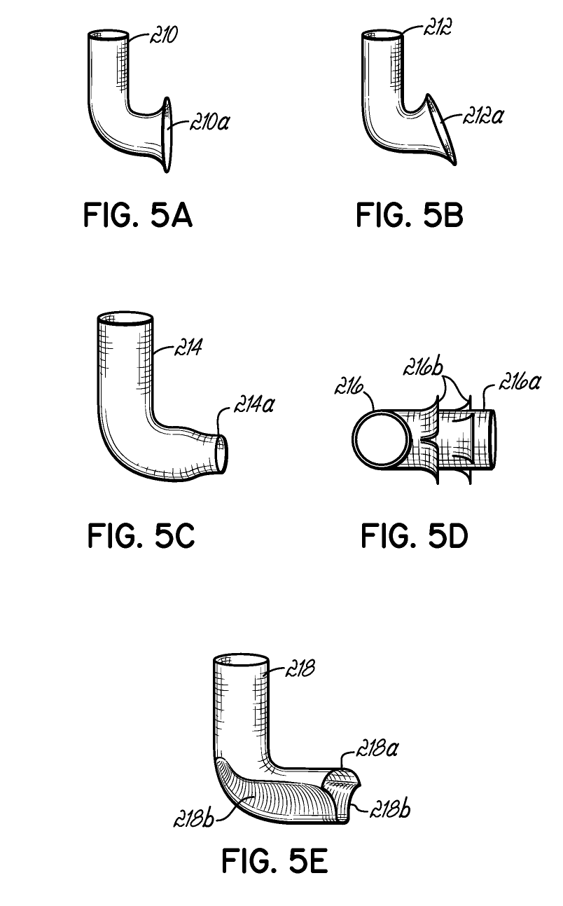

The various tubes shown and described herein may be configured in many different forms. For example, they may be formed as a stent structure, movable between contracted and expanded conditions and with a curve or bend that is either preformed or formed during the act of expanding the stent. The stent may be designed so that it is covered in the aorta and open in the arch vessel. That is, the stent structure may have a typical cover material associated with it for a portion that will be situated within the aorta and may have an open configuration, such as a mesh or other wire cage type design, for placement in the arch vessel. To prevent the tube or tubes from collapsing in the aorta due to systolic flow of blood, the stent may be designed such that it includes a stiffer lengthwise portion for residing in the aorta and a more flexible (e.g., open mesh or wire cage) portion for residing in one of the arch vessels. More generally stated, the stent may be configured with a variable strength or stiffness along its length. This may be accomplished by using a different mechanical design, such as a different wire support design along one portion of the length relative to another portion of the length and/or different material compositions for one portion of the length relative to another portion of the length. As another manner of preventing the tube or tubes from collapsing within the aorta, support members could be used to extend between outer walls of the tube or tubes and the inner wall of the aorta. The tube or tubes could also be designed in other manners that cause them to be well supported by the aorta itself. For example, the tube or tubes could have a support feature or features similar to those discussed below in connection with FIGS. 8A-8C, 9A-9B or 10A-10B. In another potential variation, a portion of the tube or tubes that is/are configured to reside in the aorta may have a flange that abuts with the inner wall of the aorta (e.g., a disc extending around the tube), or a wider portion of the tube that is formed similar to a dumbbell shape or a locally dilated circumferential segment along the length of the tube for purposes of engaging the aortic wall and adding overall strength to the tube. Individual tubes may be linked together for added support. Hemodynamically shaped front or upstream ends or sides may be used to lessen the impact forces as blood flows against the tube or tubes. To prevent multiple tubes within the aorta from colliding with each other, the tubes may be formed with bends or curves away from one another when situated within the aorta.

FIGS. 8A and 8B illustrate another embodiment of a device 270 including a deflector element 272 coupled to mounting structure 274. In this embodiment, the mounting structure 274 is also stent-like and engages the inner wall of the aorta 20 to secure the device in position as illustrated in FIG. 8A. Again, as with all other embodiments, any suitable mounting structure may be used instead, such as barbs, hooks, adhesive or any other structure or feature that will adequately secure the device within the aorta 20. The deflector element 272 provides an overhang generally in line with the entrance to one or more of the arch vessels 22, 24, 26 for physically diverting the blood flow and any particles or other emboli therein. The deflector element 272 will physically divert the blood flow to encourage a downward flow through the curve 20b in the aorta 20 thereby also encouraging a downward flow of any particles or other emboli therein until such time as the particles or other emboli have passed the arch vessels 22, 24, 26.

FIG. 8C illustrates that the stent-like mounting structure 274 or other mounting structure may be connected to the deflecting or deflector element 272 in a manner opposite to that shown in FIG. 8B. That is, the stent-like structure 274 is shown as connected to the outside surface of the deflecting element 272, whereas the stent-like structure or element 274 is shown to be connected to the inside surface of the deflecting element in FIG. 8B. It will be appreciated that any other connection may be used instead including, for example, a connection that sandwiches the stent-like structure 274 between layers of the deflecting element 272. The stent-like element 274 may be placed into contact with tissue of the aorta 20 such that it becomes essentially embedded into the aortic wall as tissue grows into it.

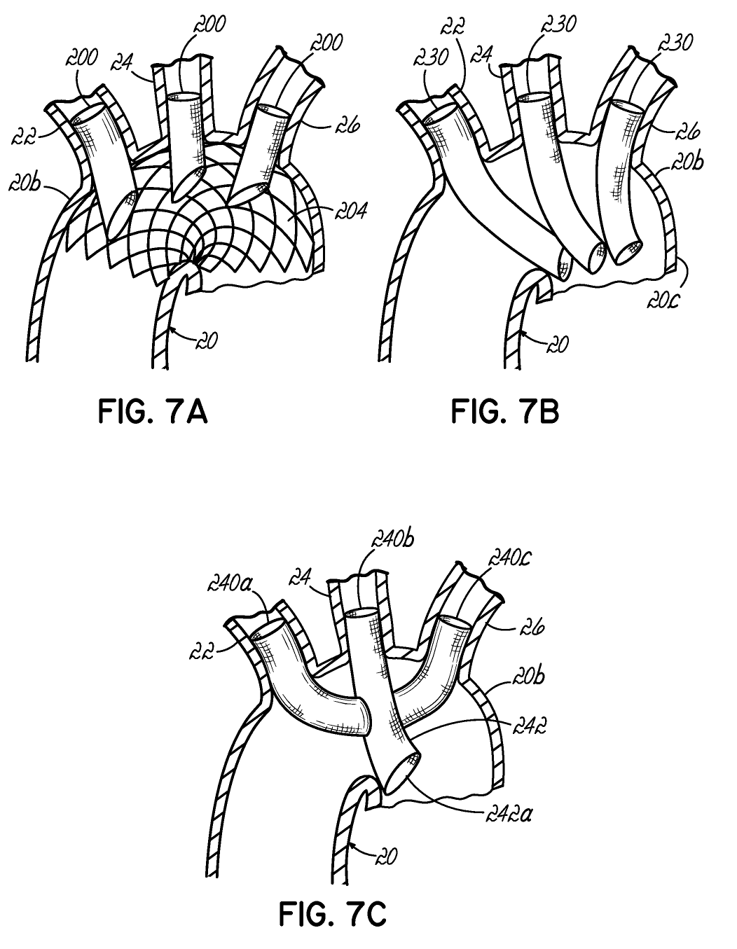

FIG. 9A illustrates another alternative embodiment of a deflector device 280. This embodiment illustrates a hybrid of a pipe or tube portion 282a contained in the arch vessel 22 and an overhang 282b situated within the aorta 20 to provide protection to the first arch vessel 22 and the next two arch vessels 24, 26. As one of many possible alternatives, the overhang portion 282b′ shown in FIG. 9A may be reconfigured to essentially form a closed space similar to that shown in FIG. 3A and illustrated in device 280′ of FIG. 9B. This would create an upward opening into the second arch vessel 24, while still allowing blood flow through the tubular portion 282a into the first arch vessel 22. Mountings, such as the stent-like mounting structures 284a, 284b or other structures, may be used within the arch vessel 22 and within the aorta 20, or within either the arch vessel 22 or the aorta 20. The embodiments shown in FIGS. 9A and 9B have various advantages, such as requiring less foreign material inside the aorta 20, less risk of clot formation on the material, less risk of migration or shifting of the device 280 and ease of implantation. In this latter regard, the operator would only have to enter one of the arch vessels 22, 24, 26 and then deploy the device 280, for example, from a suitable catheter or other deployment device or surgical tool.

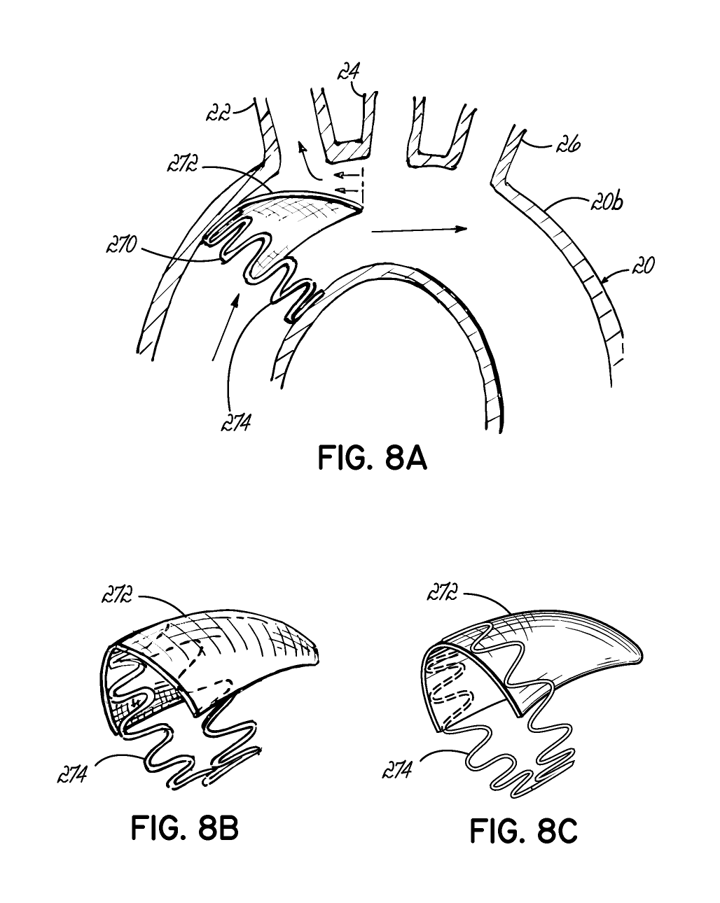

FIGS. 10A and 10B illustrate another embodiment of a deflector device 290 which may be mounted with a stent 294 and serves to isolate one or more of the arch vessels 22, 24, 26 from blood flow. Blood flow is then provided from the distal aortic arch or descending aorta 20c into an inflow tube 292. The inflow tube connects with or communicates with an isolation element or shield 296 that creates a space for directing the blood to the arch vessels 22, 24, 26. The blood entering the inflow tube 292 would be much less likely to contain one or more particles or other emboli since the emboli would tend to continue traveling down the aorta 20 as opposed to reversing flow direction into the inflow tube 292. This device 290 may be configured differently for open surgical procedures. For example, the arch vessels 22, 24, 26 may be perfused by an inflow graft taken from a part of the aorta 20 where the risk of embolic material entering is low. This could be in the descending aorta 20c where the graft would lead back up to the arch 20b, or from the side of the aorta and, more particularly, the inside of the arch 20b or at a lower location in the ascending aorta 20a perhaps near the coronary arteries (not shown) very low in the aorta 20. For example, grafts exist for replacing the arch vessels 22, 24, 26 where the arch vessel branches take off from the outer curve of the graft.

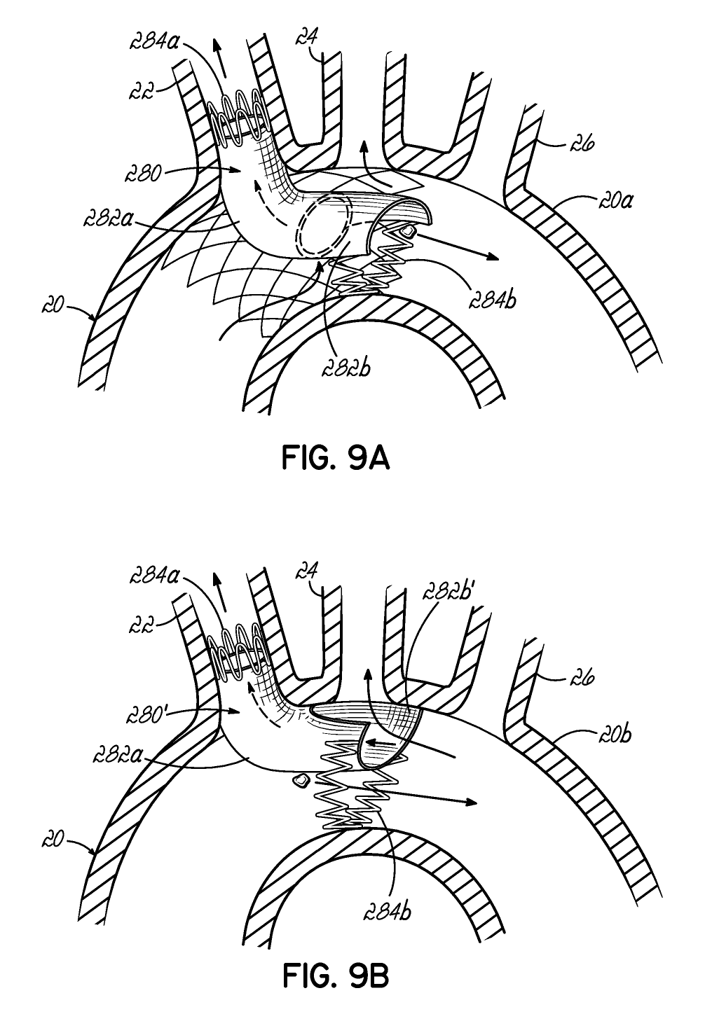

FIGS. 11A and 11B illustrate another embodiment of a device 300 including a shield 302 coupled to a mounting structure 304 again in the form of a stent-like structure. Again, the stent-like structure may be substituted with any other suitable surgical or catheter deployed mounting structure, including grafts or other manners of securing structures within vessels such as the aorta 20. In this embodiment, a shield member 302 is used having an inflow at least at one end thereof. For example, in FIG. 11A only a single inflow end opening 302a is shown in the ascending aorta 20a at an outer or peripheral location of the blood flow where emboli may be less likely to be included in the flow. FIG. 11B illustrates inflow openings 302a, 302b at opposite ends of the shield 302 to also allow retrograde blood flow into the space 302c communicating with each of the arch vessels. The opening 302b communicating with the descending aorta 20c may be desirable or important for allowing additional blood flow that has a low risk of emboli and allowing a catheter procedure from the groin to evaluate the arch vessels 22, 24, 26 and the shield 302. Another variation (not shown) may include an inflow from the aortic root that directs blood in a conduit to the space 302c leading to the arch vessels 22, 24, 26 or to one or more separate outflows communicating with the arch vessels 22, 24, 26. The inflow may be a full circular structure or a partial hoop or circular structure that encompasses all or part of the aortic root to draw perfusing blood directly from a location that has a low risk of containing emboli.

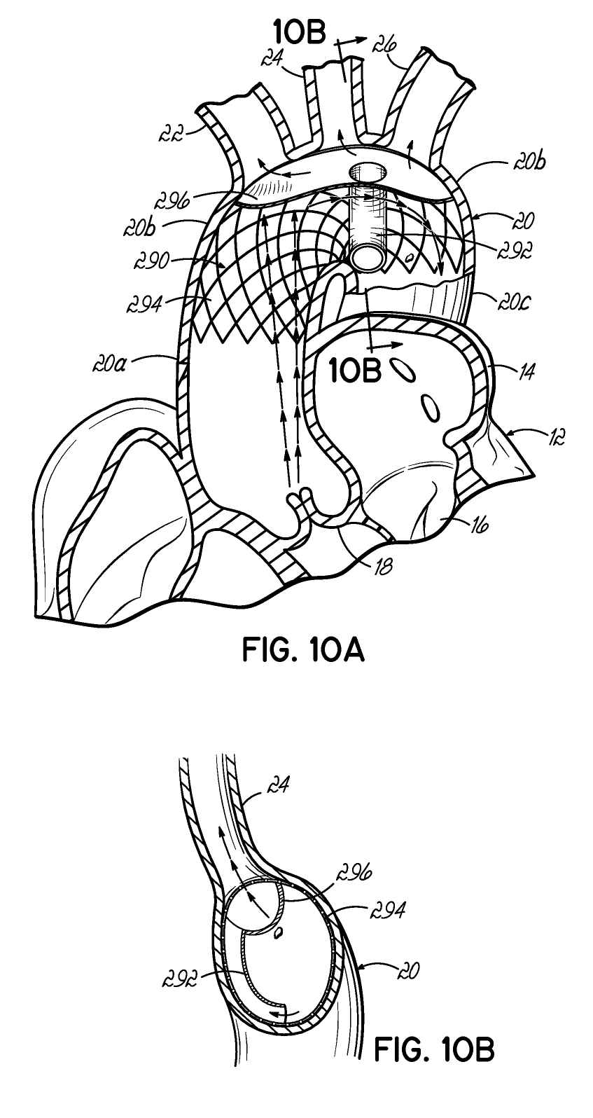

FIG. 12 illustrates a deflecting device 320 incorporating a shield member 322 having an inflow end or portion 322a located low in the ascending aorta 20a relatively near to the aortic root, but not interfering with the operation of the aortic valve 18. As mentioned above, this location in the aorta 20 may be less likely to contain emboli due to the velocity profile of blood flow in the aorta 20. For example, the coronary arteries (not shown) take off from the aorta 20 in this region and have much lower incidence of receiving emboli than the arch vessels. The distal end 322b of the device 320 is shown closed, but the distal end 322b could also be open to allow blood to flow backwards or in retrograde fashion to the entrances 22a, 24a, 26a of the arch vessels 22, 24, 26 and to permit angiographic study from the groin. Device 320 is illustrated with a stent type mounting structure 324.

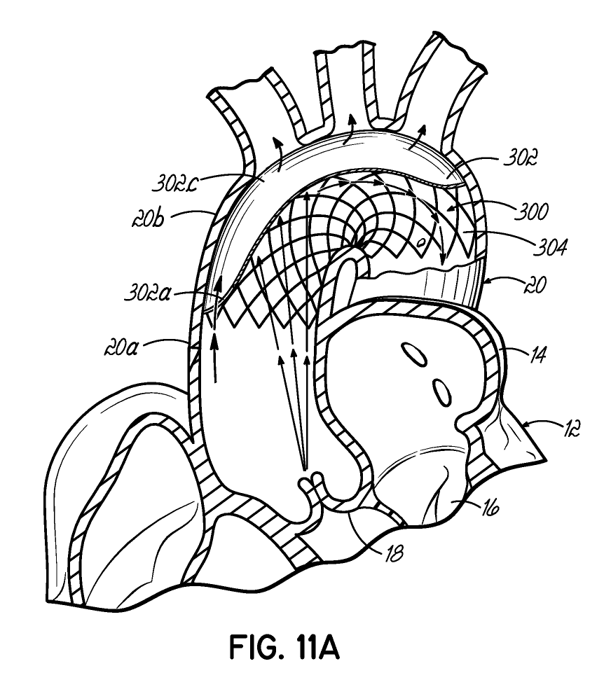

The variation shown in FIG. 13 is a device 330 having a shield 332 with a tubular flow channel for providing less obstruction of blood flow in the aorta 20. This shows a larger channel or tubular structure than that shown in FIG. 12, and includes an open distal end 332a. Central blood flow through the aortic valve 18 travels through the flow channel 332 and down into the descending aorta 20c to the lower portions of the patient's body. Peripheral blood flow travels in the direction of arrows 336 to the arch vessels 22, 24, 26 outside of channel 332. Device 330 is again illustrated with a stent-like mounting structure 334 as an example.

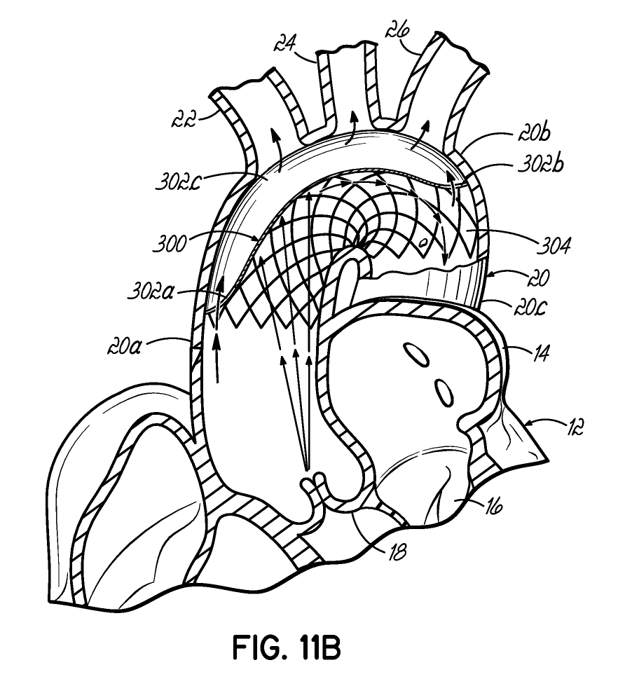

FIGS. 14A and 14B illustrate another embodiment of a deflection device 350 having a tubular flow channel structure 352 positioned more centrally in the aorta 20 and through the turn 20b in the aorta 20. This device 350 will allow central blood flow that may be more likely to contain emboli to flow into the tube 352 and out into the descending aorta 20c while also allowing full blood flow around the tube 352 and past any suitable mounting structure 354 used to mount the tube 352 generally centrally within the aorta 20 and into the arch vessels 22, 24, 26. Again, any suitable mounting structure may be used so long as the mounting structure allows blood flow around the tubular structure 352 and into the arch vessels 22, 24, 26 while diverting particles or other emboli into a blood flow path that ensures they are carried downwardly into the descending aorta 20c and past the entrances 22a, 24a, 26a to the arch vessels 22, 24, 26.

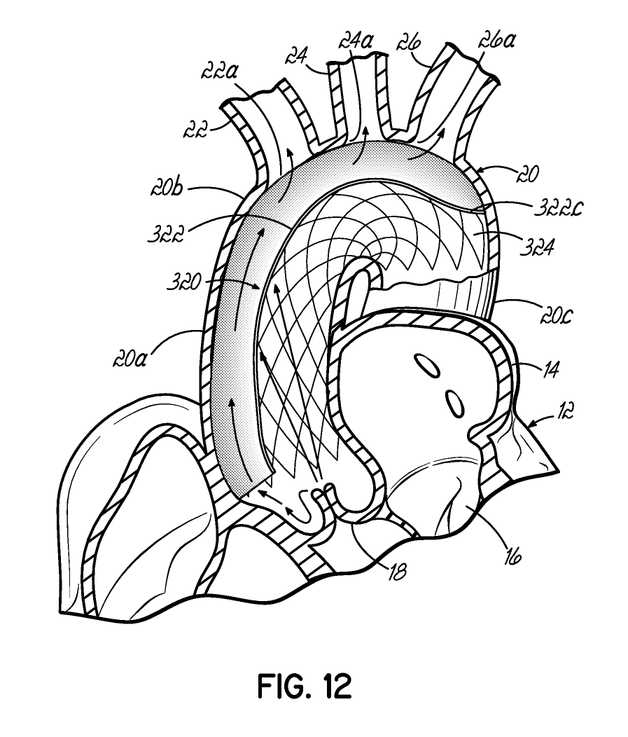

FIG. 15 is an illustrative view schematically illustrating a likely path of emboli 360 in the blood flow with a theoretical blood flow velocity profile indicated low in the ascending aorta 20a and generally at the curvature of the aortic arch 20b. In this regard, emboli in the high velocity central flow low in the ascending aorta 20a will be directed upwardly to the outer, upper wall of the aorta 20 and directly into one of the arch vessels 22, 24, 26.

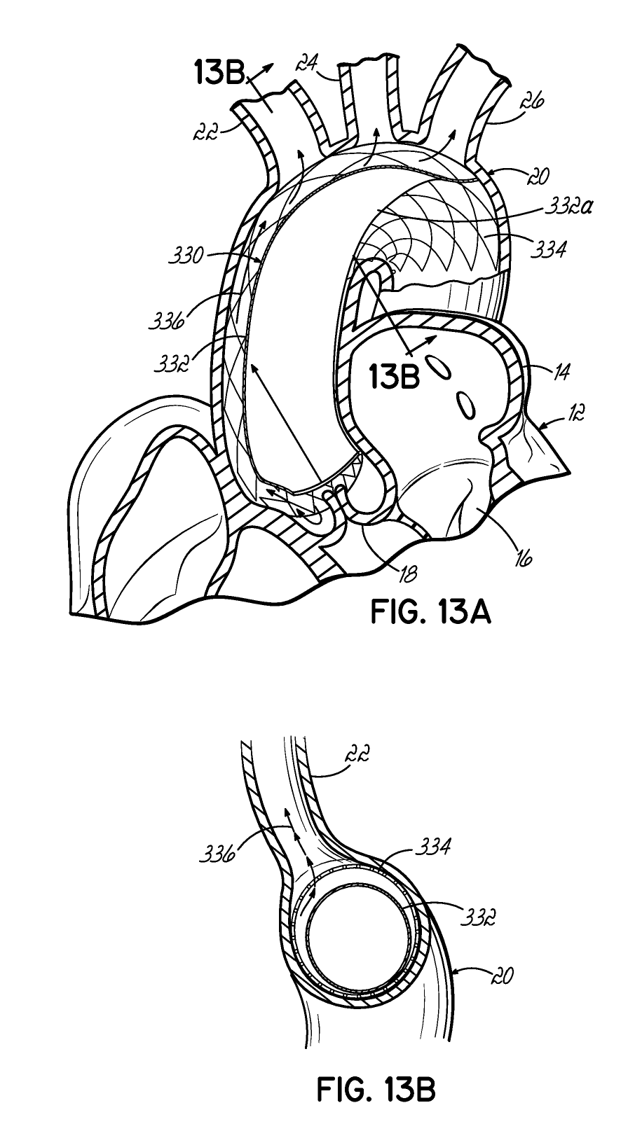

FIG. 16 is a view similar to FIG. 15 but indicating a darker colored region showing the likely regions of higher velocity blood flow through the aorta 20 and into the arch vessels 22, 24, 26.

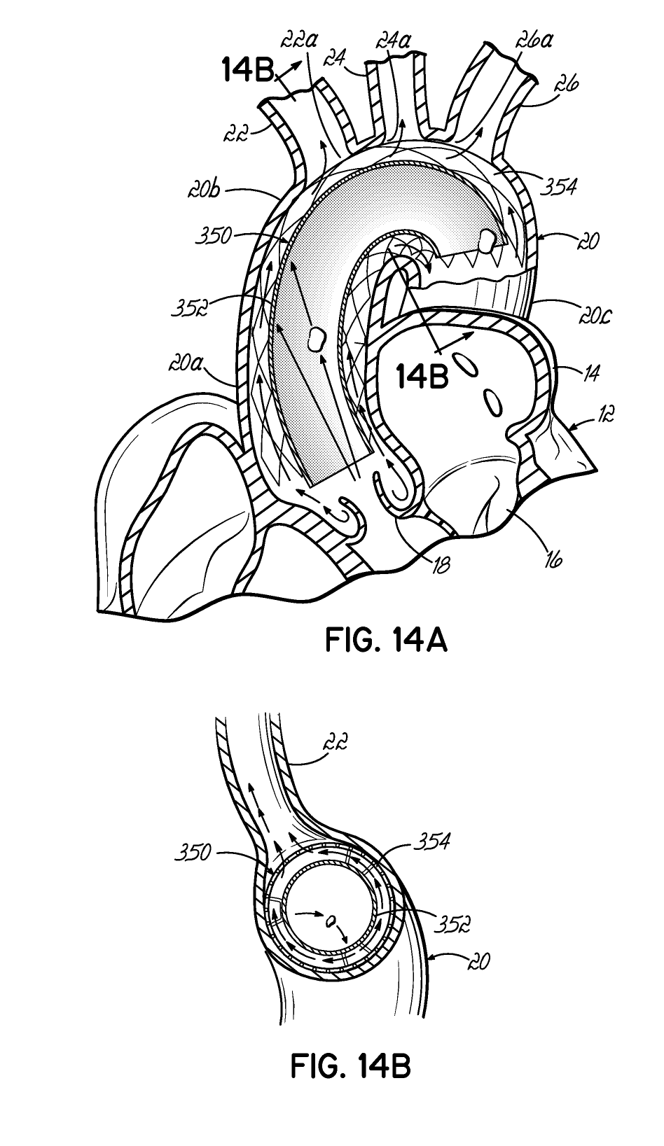

FIG. 17 illustrates a graft 400 that has three branches 402, 404, 406 for the three separate arch vessels 22, 24, 26. FIGS. 17-20, 21A and 21B show various forms of grafts that may or may not be used in conjunction with a supporting stent structure. FIG. 17 illustrates a conventional graft 400 having a portion that forms the aortic arch and respective tubular portions 402, 404, 406 that connect with the arch vessels 22, 24, 26. FIG. 18 illustrates a graft device 410 directing reversed or retrograde blood flow from the descending aorta 20c to the arch vessels via a tube 412 branching into three separate tubular portions 412a, 412b, 412c that connect with the respective arch vessels (not shown). Although device 410 is illustrated with a conventional zig zag type wire support structure 410a there may be no need for such support in a surgical graft embodiment as disclosed herein. Again, aortic stent grafts with or without support structures, such as wire configurations, may be used as the mounting structure in any of the embodiments disclosed or otherwise encompassed by the present disclosure.

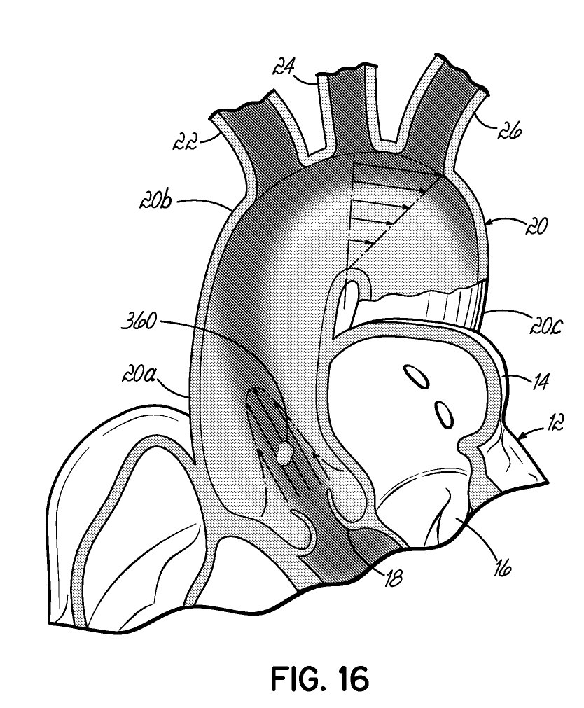

FIG. 19 illustrates another embodiment of a graft device 420 in which the inflow is taken from the inside of the aortic arch. At the inside of the aortic arch, there is a lower chance of emboli in the blood flow. This embodiment illustrates three separate tubes 422, 424, 426 that would connect separately to the three arch vessels (not shown). It will be understood that a single tube may connect to the inside location of the aortic arch and then branch into three tubular portions connecting with the respective arch vessels.

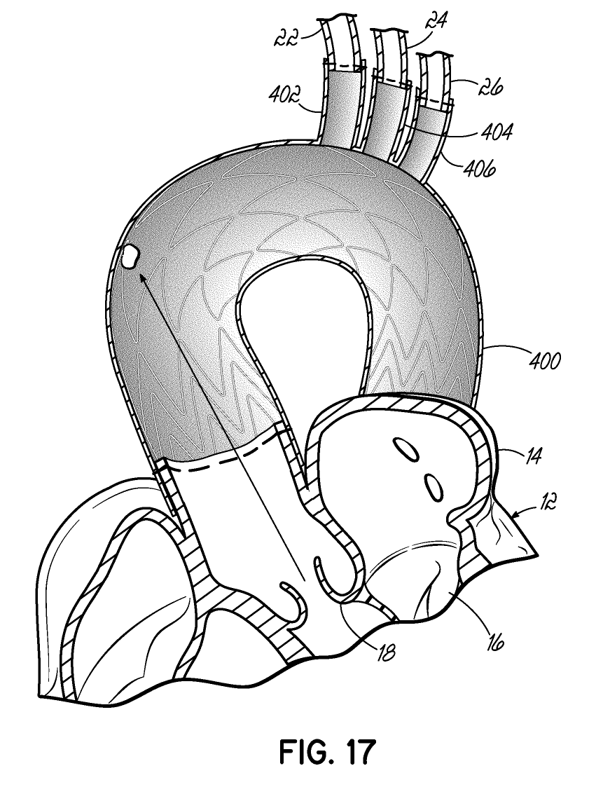

FIG. 20 illustrates an embodiment of a device 430 similar to FIG. 19, but illustrating that the inflow tubes 432, 434, 436 may lead to a location low in the ascending aorta. This region is near the origins of the coronary arteries where the risk of embolism is believed to be low.

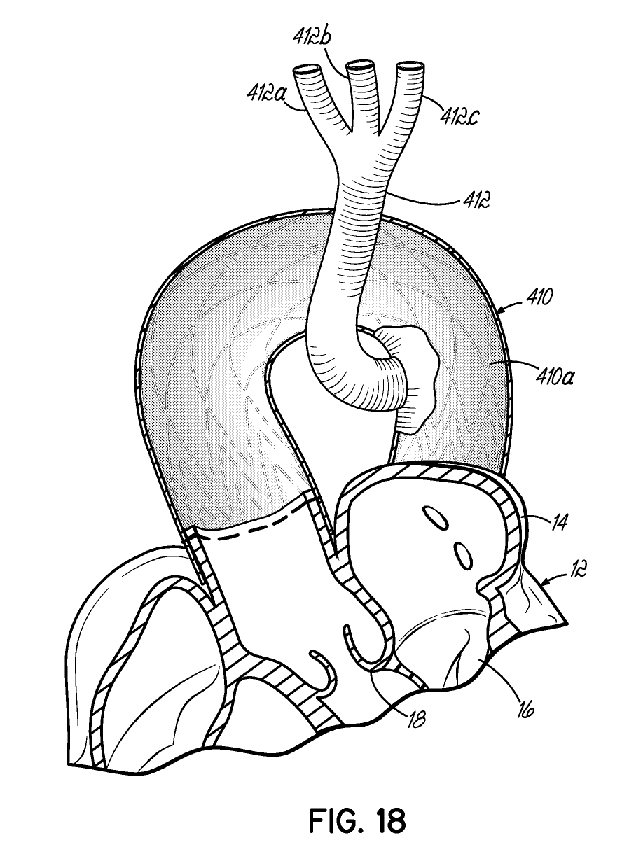

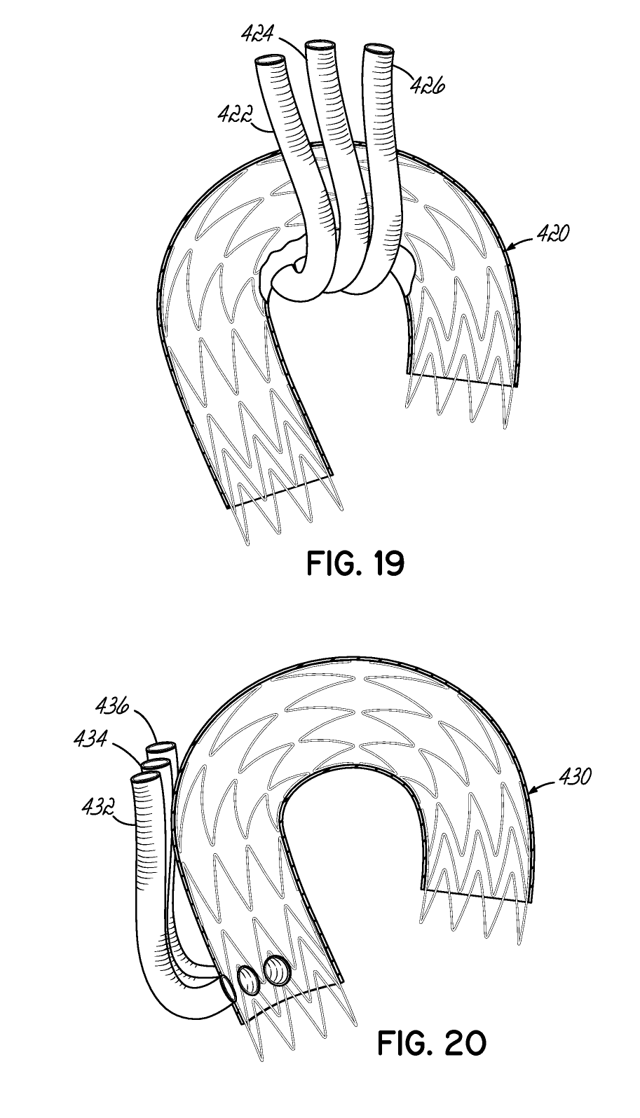

FIG. 21 illustrates another embodiment of a device 440 showing aortic graft with an enlargement 442 simulating the natural sinus of valsalva, i.e., the area behind the aortic valve leaflets. FIG. 21 illustrates a fully circumferential enlargement 442, while another alternative device 440′ illustrated in FIG. 22 shows that the enlargement 442′ need not be completely circumferential. Again, the embodiments of FIGS. 21 and 22 take blood inflow into tubes 444, 446, 448 from low in the aorta 20 proximate the aortic valve leaflets where the risk of emboli in the blood is lower. Tubes 444, 446, 448 would be connected to the arch vessels (not shown).

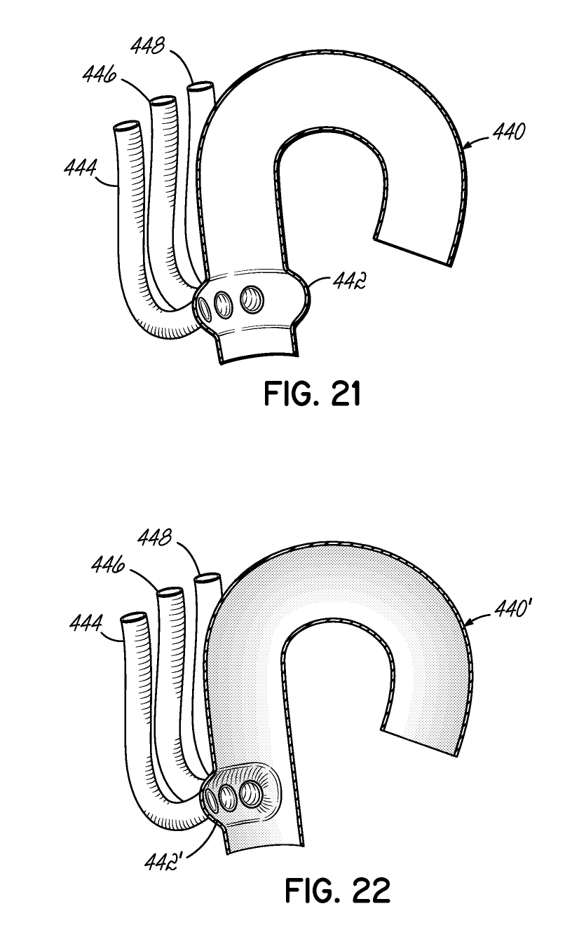

FIG. 23 illustrates another embodiment of a device 470 that involves adding one or more valve structures 472 in a position downstream from the native aortic valve 18. This can take advantage of the flow dynamics associated with the native aortic valve 18 but at a location proximate to the entrances 22a, 24a, 26a of the arch vessels. Valve structures 472 are schematically illustrated to appear similar to the native aortic valve 18 and may include one or more movable valve elements, such as one or more movable flaps to act in a similar manner to a one way check valve. If the valve structures 472 are constructed to operate, e.g., open and close, in a manner similar to the native aortic valve 18, then a blood flow velocity profile similar to the profile created by the native aortic valve 18 may be established at one or more locations proximate to at least one of the entrances 22a, 24a, 26a. The higher velocity central flow is more likely to contain emboli and, therefore, emboli is directed past and away from the entrances 22a, 24a, 26a to the arch vessels 22, 24, 26 while the lower velocity blood flow at radially outward or peripheral locations of the valve structure(s) 472 supplies blood flow into the arch vessels 22, 24, 26. Thus, one or more valve structures 472 as schematically illustrated in FIG. 23 may be placed at the arch 20b of the aorta 20 to direct the high velocity flow to the radial center of the arch 20b thereby encouraging emboli to follow a radially central blood flow path through the curve or arch 20b of the aorta 20 and downwardly into the descending aorta 20c as opposed to following a path on the periphery of the aorta 20 and potentially into the arch vessels 22, 24, 26. It may not be necessary for this type of valve structure 472 to completely close, as does the native aortic valve 18. It may be useful to allow retrograde blood flow to occur as this is generally how coronary flow occurs. A valve that completely closes may limit the flow of blood to the coronary arteries. One or more valves 472 may be used anywhere in the region of the aortic arch 20b and may be mounted in any suitable manner such as the stent-like structure 474 shown in FIG. 23, or any other structure or features.

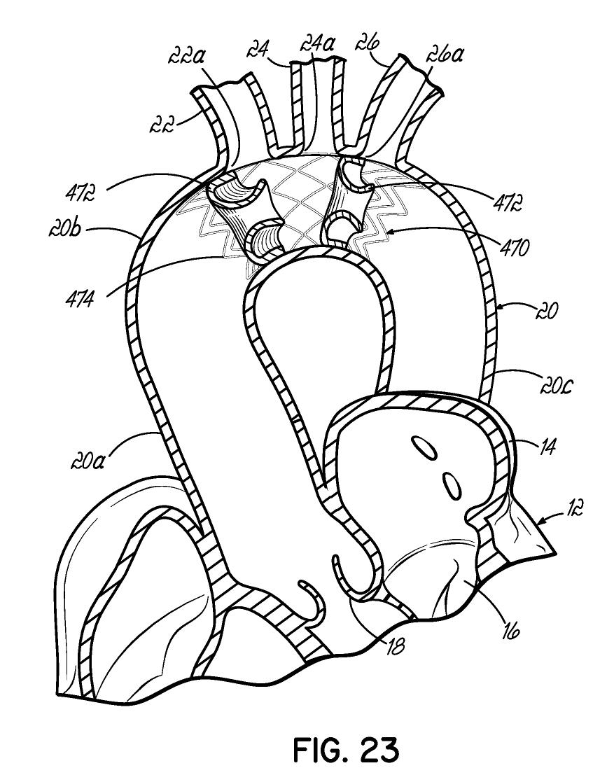

FIG. 24 illustrates another embodiment of a device 490 similar to FIG. 23, but adding a central conduit or tubular structure 492 to further direct emboli into the descending aorta 20c and past the arch vessels 22, 24, 26. Again, a stent-like mounting structure 494 is shown for illustration purposes. In this device, a valve 472 may be used at the inlet of the device 490 to centralize high velocity peripheral flow that more likely will contain any emboli. Lower velocity flow that is less likely to contain emboli flows around the tubular structure 492 as shown by the arrows 496 and into the arch vessels 22, 24, 26. Openings may be provided at the proximal and/or distal ends of the device 490 to allow this peripheral, lower velocity blood flow around the tubular structure 492 and into the arch vessels 22, 24, 26. The conduit is located low and close to the valve 472 so that the flow moving around the conduit 492 makes at least two turns in a generally S-shaped path that would be difficult for a particle or other emboli 498 to follow. The blood flow on the outside of the conduit or tubular structure 492 would therefore be more likely free of particles or other emboli 498. This device 490 may be mounted on a conduit introduced percutaneously and deployed near the arch 20b, or again like any of the other embodiments, implanted in another type of surgical operation.

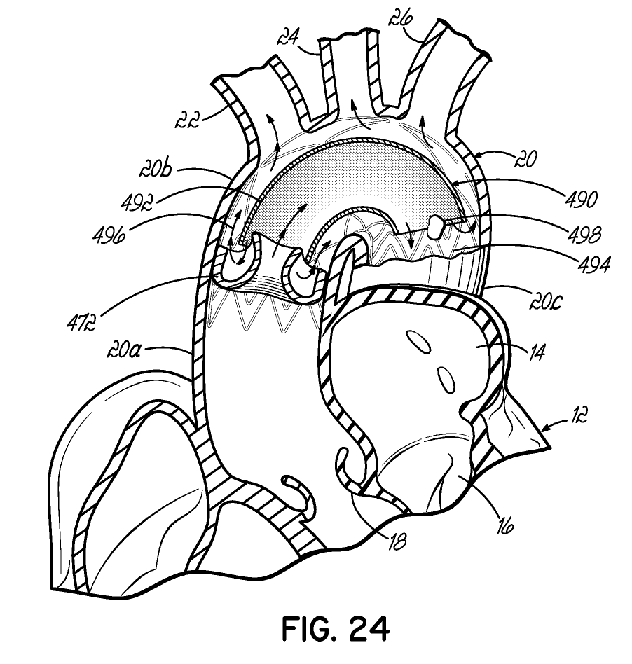

FIGS. 25A and 25B demonstrate how catheters can be used to insert these devices as well as procedural methods important in avoiding embolization during device deployment. A catheter 520 is shown delivering a stent 522 (which is impervious to the passage of emboli at least in the part to be positioned inside the body of the aorta 20). The stent 522 shown is self-expanding (similar to stents used in carotid procedures) but it could be balloon expandable instead, for example. It is very common for atherosclerotic material 524 to reside around the orifice or entrance 22a, 24a, 26a of an arch vessel 22, 24, 26 as it arises from the aorta 20. The material is frequently located around the periphery of the take off of the vessel from the aorta, but frequently also extends into the vessel. Manipulation of this area may dislodge debris which can then pass into the brain. By delivering a stent 522 into a vessel supplying the brain, beyond this region highly subject to disease, the stent 522 can initially avoid the area of disease. The stent 522 can then be partly deployed so that it “occludes” (at least temporarily) the flow to the brain. When the stent is deployed more proximally, debris that is dislodged from a plaque cannot pass into the head as it will be crushed under the stent 522. Any loose material can then pass distally into the lower part of the body and avoid the brain. The sequence of application of the devices (e.g., tubes or stents 522) should minimize the risk of embolization. It can be desirable, where possible, to place devices in vessels that are more distal (i.e., downstream relative to the direction of blood flow) before adding devices to more proximal vessels. In FIG. 25B, the subclavian vessel 24 is treated first and then the more proximal left carotid vessel 26 is treated second. If debris is dislodged in treating the middle arch vessel 24, it is unlikely to pass into the left subclavian vessel 26.

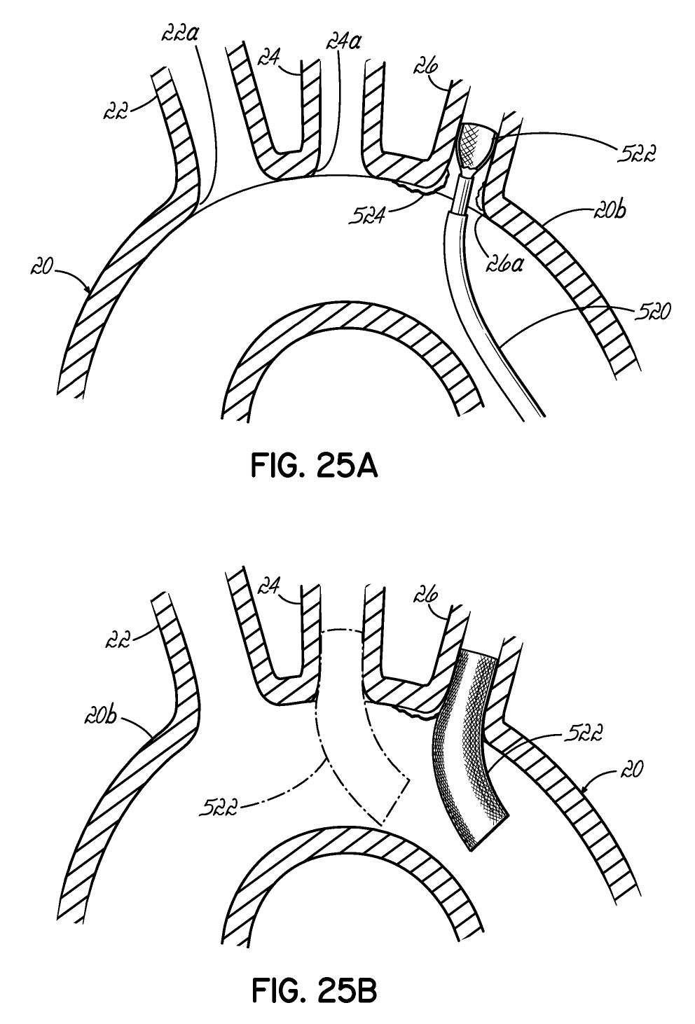

FIG. 26 demonstrates the use of an embolic protection device 554 inserted in an arch vessel 22. As described previously, the area around the take off or entrance 22a of an arch vessel 22 often is quite diseased. If an upstream vessel requires treatment prior to a more distal vessel or if a distal embolic divert will make insertion of a more proximal device more difficult, then it will be advisable to prevent any loose debris from entering more distal arch vessels. In FIG. 26 a diverter or deflector element or device 550 is being positioned in the brachiocephalic (or innominate) artery 22. A fragment of debris 552 is shown being dislodged and carried more distal with the flow of blood. To prevent the material 552 from passing into the brain, a protection device 554 is placed over the next two arch vessels 24, 26. The protection device 554 is shown being delivered from a catheter 556. The device 554 must not allow debris 552 to pass. It can be totally impervious to blood (in which case flow to the brain may be temporarily occluded) or the device 554 may permit the passage of blood but not larger materials. The brain will tolerate short periods of reduced blood flow without permanent damage, so a protection device 554 can fully or partly reduce the flow of blood without serious consequence. The protection catheter 556 can be removed after the procedure is complete.

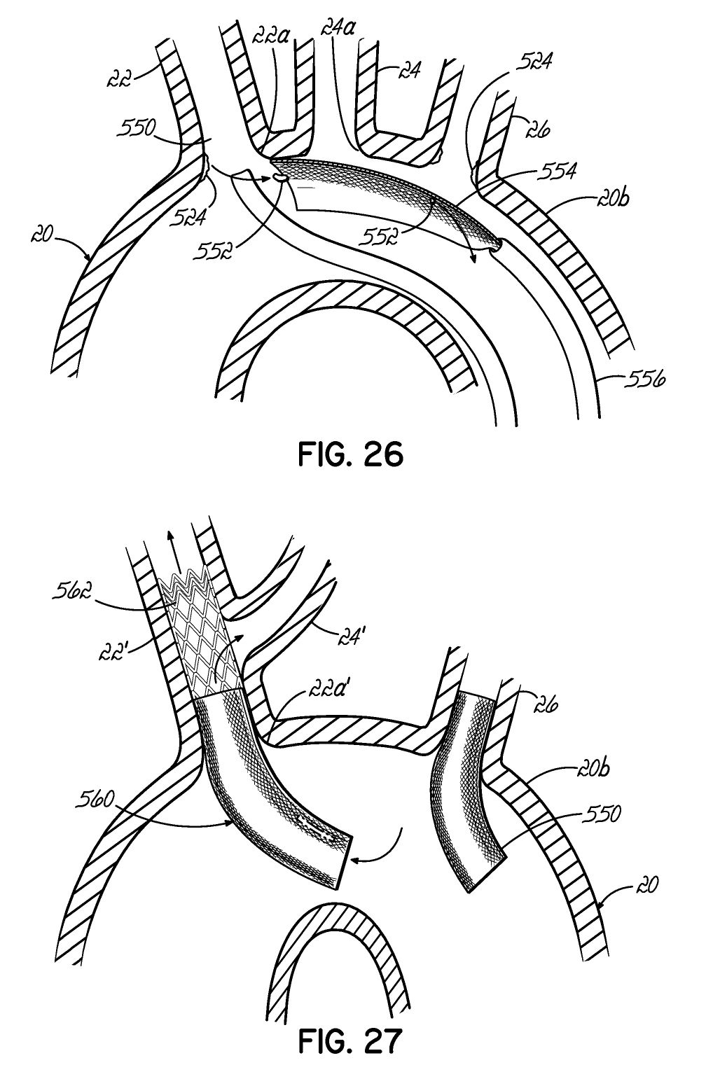

There are many normal variants in the pattern of branching of brain vessels. In the vast majority, the aortic arch gives rise to three vessels 22, 24, 26 as described herein. One reasonably common variation is shown in FIG. 27 where only two vessels 22′, 26 arise from the aorta 20 (e.g., the pattern seen in a cow and thus often referred to as the bovine aorta pattern). In this situation, the left carotid 24′ takes its origin from the innominate artery 22′ beyond the take off or entrance 22a′. Embolic protection devices shown previously may obstruct the flow to this left carotid branch 24′. Thus, a deflector device 560 is shown that allows flow to this side branch 24′ as indicated by an arrow directed through an open portion 562 of the device 560. In general, deflector elements described here are only required to be impervious to emboli in the portion inside the aorta 20 (i.e., it is not necessary to be impervious in the inside of the target arch vessel). There are many ways to accomplish this objective. The stent can be covered, or the stent can be impervious to blood by its manufacture, or the stent can have a plastic or other material closing the space between parts of the stent.

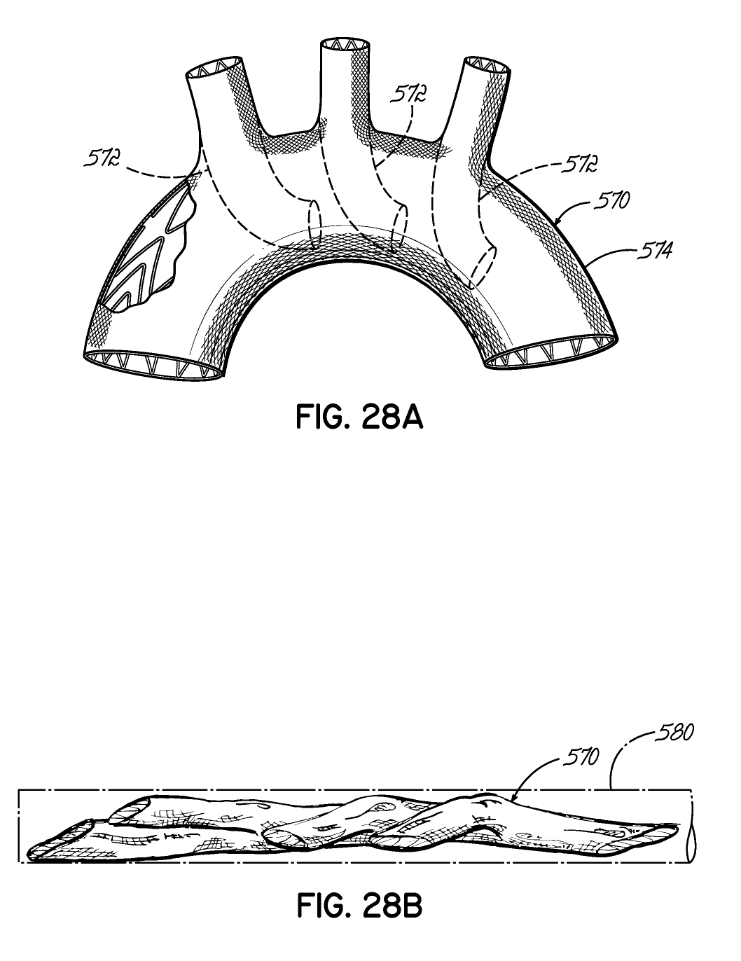

In some patients the entire aorta is heavily diseased and is an ongoing risk of brain emboli. In fact, a heavily diseased aorta has been correlated with a decline in mental function in the elderly. It is highly probably that in these patients, repeated episodes of embolization results in recurring brain injury that causes mental decline. The device 570 shown in FIGS. 28A and 28B combines an embolic deflector (shown here as tubes 572 but capable of substitution by any other suitable configurations such as those shown previously), and a tube graft 574 that relines the entire aorta. This will exclude the aorta from flow where it is covered by the device 570. Thus, material from this region cannot embolize because it is trapped beneath the graft 574. Emboli from other sources will be diverted.

FIG. 28B demonstrates how this device 570 could be contained in a catheter 580 for insertion. This device could then be passed into the arterial system and advanced near the arch of the aorta, and then deployed. To ensure that the branches of the device enter the arch vessels appropriately, it may be useful to pass guidewires through each of the branches or tubes 572 of the embolic protection device 570 and then direct these into the appropriate target vessel. These guidewires can then direct each portion or limb of the device 570 into the appropriate branch vessel. The individual branches 572 of the device 570 may instead be delivered in individual sheaths or catheters. Another useful variation would be to have the branches 572 of the device 570 that are placed inside the arch vessels involuted inside the aortic graft. The main aortic covering component could be inserted first into position. The branches 572 of the device 570 would first sit inside the main tubular portion and then could be inverted or extended outwardly to their final position inside the arch vessels. This may simplify insertion.

It is also possible to involute the portion of the deflector device 570 sitting inside the aorta in the part of the component that sits inside the branch vessel for placement. The aortic component could then be turned inside out and allowed to sit inside the aorta. This could be done separately (e.g., the tubular deflectors could be placed individually like this) or in conjunction with a device 570 such as shown in FIG. 28. Another option would be construct this device 570 in-situ. An arch graft could be advanced into the aorta with holes precut or cut in-situ. The embolic protection component could then be added by advancing brain protection elements.

Similar functional results could be achieved by first placing individual tubes in each of the arch vessels (as shown in FIG. 25 and others). A cover graft could then be placed in the ascending aorta and arch of the aorta. The tubes must be long enough to prevent the aortic stent from occluding. This is essentially combining this idea with the tube shown in FIG. 28A and may be referred to as a crush technique with two stents in one channel.

While the present invention has been illustrated by a description of various preferred embodiments and while these embodiments have been described in some detail, it is not the intention of the Applicant to restrict or in any way limit the scope of the appended claims to such detail. Additional advantages and modifications will readily appear to those skilled in the art. The various features of the invention may be used alone or in any combination depending on the needs and preferences of the user. This has been a description of the present invention, along with the preferred methods of practicing the present invention as currently known. However, the invention itself should only be defined by the appended claims.

What is claimed is:

1. A device for preventing stroke due to embolic material in the bloodstream of a patient, the patient having an aorta with an ascending portion and a descending portion, and one or more arch vessels communicating with the aorta for directing blood flow to the brain of the patient, the device comprising:
a physical deflector element configured for at least partial placement in the aorta of the patient, and
mounting structure coupled to the physical deflector element, the mounting structure configured to engage at least one of the aorta or an arch vessel communicating with the aorta,
wherein the physical deflector element is constructed and arranged to direct blood flow in the aorta in a manner that directs embolic material in the blood flow past the one or more arch vessels and into the descending portion of the aorta.

2. The device of claim 1, wherein the physical deflector element further comprises at least one generally tubular member.

3. The device of claim 2, wherein the generally tubular member is curved to generally follow an arch in the aorta.

4. The device of claim 2, wherein the generally tubular member is configured for mounting within one of the arch vessels communicating with the aorta such that a portion of the generally tubular member extends within the aorta.

5. The device of claim 2, wherein the generally tubular member further comprises multiple tubular portions.

6. The device of claim 2, wherein the generally tubular member includes at least one non-tubular portion.

7. The device of claim 1, wherein the physical deflector element further comprises a shield member constructed and arranged to shield at least one entrance to an arch vessel and redirect blood flow away from the entrance.

8. The device of claim 7, further comprising a generally tubular member coupled with the shield member, the generally tubular member allowing retrograde flow of blood to the entrance of the arch vessel.

9. The device of claim 1, wherein the physical deflector element further comprises a ramp member.

10. The device of claim 1, wherein the physical deflector element further comprises at least one element that causes a generally spiral or whirling blood flow in the aorta.

11. The device of claim 1, wherein the physical deflector element further comprises a plurality of shield elements.

12. The device of claim 1, wherein the physical deflector element further comprises a flow restricting element.

13. The device of claim 12, further comprising a generally tubular member coupled with the flow restricting element.

14. The device of claim 13, wherein the generally tubular member further comprises a blood flow inlet and a blood flow outlet, and the flow restricting element is mounted closer to the blood flow inlet than to the blood flow outlet.

15. The device of claim 1, wherein the mounting structure further comprises a stent-like structure for engaging an inner wall of an arch vessel or an inner wall of the aorta, or both the inner wall of the arch vessel and the inner wall of the aorta.

16. The device of claim 1, wherein the mounting structure further comprises at least one of: a stent-like structure, hooks, barbs, spring elements, adhesive, suture, fabric or an aortic graft.

17. The device of claim 1, wherein the physical deflector element is collapsible for delivery through the arterial system of the patient and expandable for deployment at least partially in the aorta.

18. The device of claim 1, wherein the physical deflector element further comprises a tubular aortic graft constructed and arranged to replace a portion of the aorta.

19. The device of claim 18, further comprising at least one tubular arch vessel graft coupled with the aortic graft and configured to supply blood flow to at least one arch vessel of the patient.

20. A device for preventing stroke due to embolic material in the bloodstream of a patient, the patient having an aorta with an ascending portion and a descending portion, and one or more arch vessels communicating with the aorta for directing blood flow to the brain of the patient, the device comprising:
a generally tubular physical deflector element configured for placement in the aorta of the patient, the generally tubular physical deflector element having an entrance for receiving blood flow from the ascending portion of the aorta and an exit for directing blood flow into the descending portion of the aorta,
mounting structure coupled to the generally tubular physical deflector element, the mounting structure configured to engage the aorta and/or an arch vessel communicating with the aorta, and
a flow restricting element mounted to the generally tubular deflector element and configured to direct a first portion of the blood flow through the entrance and a second portion of the blood flow around the generally tubular physical deflector element to the one or more arch vessels.

21. The device of claim 20, wherein the generally tubular physical deflector element is curved to generally follow an arch in the aorta between the ascending portion and the descending portion.

22. The device of claim 21, wherein the mounting structure further comprises at least one of: a stent-like structure, hooks, barbs, spring elements, adhesive, suture, fabric, or an aortic graft.

23. A method of physically directing embolic material in blood flow within the aorta away from an arch vessel communicating with the aorta, the method comprising:
mounting a physical deflector element at least partially within the aorta,
using the physical deflector element to direct a first portion of the blood flow past an entrance of the arch vessel, and
directing a second portion of the blood flow to the entrance of the arch vessel.

24. The method of claim 23, wherein directing the second portion of the blood flow further comprises:
directing the second portion of the blood flow at least partially in a retrograde manner.

25. The method of claim 23, wherein using the physical deflector element to direct the first portion of the blood flow further comprises:
directing the first portion of the blood flow through a generally tubular member.

26. The method of claim 23, wherein using the physical deflector element to direct the first portion of the blood flow further comprises:
directing the first portion of the blood flow against a ramp member.

27. The method of claim 23, wherein using the physical deflector element to direct the first portion of the blood flow further comprises:
directing the first portion of the blood flow in a generally spiral manner through the aorta.

28. The method of claim 23, wherein using the physical deflector element to direct the first portion of the blood flow further comprises:
directing the first portion of the blood flow against a plurality of shield members.

29. The method of claim 23, wherein using the physical deflector element to direct the first portion of the blood flow further comprises:
shielding the entrance of the arch vessel to prevent the arch vessel from receiving the first portion of the blood flow.

30. The method of claim 29, wherein directing the second portion of the blood flow further comprises:
directing the second portion in a retrograde manner through a generally tubular member within the aorta.

31. The method of claim 23, wherein using the physical deflector element to direct the first portion of the blood flow further comprises:
mounting a generally tubular member within the arch vessel such that a portion thereof extends into the aorta.

32. The method of claim 23, wherein the physical deflector element further comprises a generally tubular member having an entrance end, and using the physical deflector element to direct the first portion of the blood flow further comprises:
mounting the generally tubular member such that the entrance end communicates with a peripheral portion of the blood flow in an ascending portion of the aorta generally adjacent to the aortic valve of the heart, and
directing the second portion of the blood flow into the entrance end.

33. The method of claim 23, wherein the physical deflector element further comprises a generally tubular member having an entrance end, and using the physical deflector element to direct the first portion of the blood flow further comprises:
mounting the generally tubular member such that the entrance end communicates with a central portion of the blood flow in an ascending portion of the aorta generally adjacent to the aortic valve of the heart, and
directing the first portion of the blood flow into the entrance end.

34. A method of replacing a portion of the aorta of a patient with a tubular aortic graft having at least one tubular arch vessel graft coupled thereto, the method performed in a manner that lessens the occurrence of stroke due to embolic material flowing to the brain of the patient and comprising:
replacing a portion of the aorta with the tubular aortic graft such that the tubular arch vessel graft is misaligned with an arch vessel of the patient, and
connecting the misaligned tubular arch vessel graft with the arch vessel.

35. The method of claim 34, wherein the tubular aortic graft includes an ascending portion, a descending portion, and an arch portion between the ascending and descending portions, the arch portion having an outer larger radius curved portion and an inner smaller radius curved portion, wherein the misaligned tubular arch vessel graft is connected to the inner smaller radius curved portion.

36. The method of claim 34, wherein the tubular aortic graft includes an ascending portion, a descending portion, and an arch portion between the ascending and descending portions, wherein the misaligned tubular arch vessel graft is connected to the ascending portion adjacent to an aortic valve of the heart.

37. The method of claim 34, wherein the tubular aortic graft includes an ascending portion, a descending portion, and an arch portion between the ascending and descending portions, wherein the misaligned tubular arch vessel graft is connected to the descending portion generally adjacent to the inner smaller radius curved portion of the arch.

38. A method of physically directing embolic material in blood flow within the aorta away from an arch vessel communicating with the aorta, the method comprising:
mounting a flow restricting element within the aorta downstream of the aortic valve of the patient,
directing a first portion of the blood flow through the flow restricting element and past an entrance to the arch vessel, and
directing a second portion of the blood flow to the entrance to the arch vessel.

39. The method of claim 38, wherein the first portion of the blood flow is directed at a higher velocity than the second portion of the blood flow.

40. The method of claim 38, wherein the flow restricting element is mounted to a generally tubular member and the method further comprises:
directing the first portion of the blood flow through the generally tubular member, and
directing the second portion of the blood flow around an outside of the generally tubular member.

41. A method of physically directing embolic material in blood flow within the aorta away from an arch vessel communicating with the aorta, the method comprising:
inserting a collapsed deflector element partially within the arch vessel from within the aorta, and
expanding the collapsed deflector element against an inner wall of the arch vessel such that a first portion of the expanded deflector element is within the arch vessel and a second portion of the expanded deflector element is within the aorta.

42. The method of claim 41, further comprising:
using a shield member within the aorta and downstream of the arch vessel during at least one of the inserting and expanding steps to deflect embolic material away from another arch vessel.

43. The method of claim 41, further comprising:
percutaneously performing the inserting and expanding steps using one or more catheter devices.

44. The method of claim 41, wherein the first portion of the expanded deflector element comprises at least one blood flow opening and the method further comprises:
generally aligning the blood flow opening with another vessel entrance communicating with the arch vessel.

45. A system for preventing stroke due to embolic material in the bloodstream of a patient, the patient having an aorta with an ascending portion and a descending portion, and one or more arch vessels communicating with the aorta for directing blood flow to the brain of the patient, the system comprising:
at least one catheter device,
a physical deflector element configured for at least partial placement in the aorta of the patient, and
mounting structure coupled to the physical deflector element, the mounting structure configured to engage at least one of the aorta or an arch vessel communicating with the aorta,
wherein the physical deflector element is constructed and arranged to direct blood flow in the aorta in a manner that directs embolic material in the blood flow past the one or more arch vessels and into the descending portion of the aorta, and the catheter device is used to deliver the physical deflector element and/or the mounting structure to the aorta and/or to the vessel.
